SEQUENCE LISTING (38 sequences)

SEQ ID NO: 1 - protein, 31 aa, synthetic construct
  REGION 1..31: note = Description of artificial sequence: synthetic protein
HGEGTFTSDVSSYLEEQAAKEFIAWLVKGGG

SEQ ID NO: 2 - protein, 228 aa, synthetic construct
  REGION 1..228: note = Description of artificial sequence: synthetic protein
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLG

SEQ ID NO: 3 - protein, 275 aa, synthetic construct
  REGION 1..275: note = Description of artificial sequence: synthetic protein
HGEGTFTSDVSSYLEEQAAKEFIAWLVKGGGGGGGSGGGGSGGGGSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 4 - protein, 424 aa, synthetic construct
  REGION 1..424: note = Description of artificial sequence: synthetic protein
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLGGGGGSGGGGSGGGGSHPIPDSSPLLQFGGQVRQRYLYTDDAQQTEAHLEIREDGTVGGAADQSPESLLQLKALKPGVIQILGVKTSRFLCQRPDGALYGSLHFDPEACSFRERLLEDGYNVYQSEAHGLPLHLPGNKSPHRDPAPRGPARFLPLPGLPPALPEPPGILAPQPPDVGSSDPLSMVGGSQGRSPSYAS

SEQ ID NO: 5 - protein, 470 aa, synthetic construct
  REGION 1..470: note = Description of artificial sequence: synthetic protein
HGEGTFTSDVSSYLEEQAAKEFIAWLVKGGGGGGGSGGGGSGGGGSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLGGGGGSGGGGSGGGGSHPIPDSSPLLQFGGQVRQRYLYTDDAQQTEAHLEIREDGTVGGAADQSPESLLQLKALKPGVIQILGVKTSRFLCQRPDGALYGSLHFDPEACSFRERLLEDGYNVYQSEAHGLPLHLPGNKSPHRDPAPRGPARFLPLPGLPPALPEPPGILAPQPPDVGSSDPLSMVGGSQGRSPSYAS

SEQ ID NO: 6 - protein, 181 aa, synthetic construct
  REGION 1..181: note = Description of artificial sequence: synthetic protein
HPIPDSSPLLQFGGQVRQRYLYTDDAQQTEAHLEIREDGTVGGAADQSPESLLQLKALKPGVIQILGVKTSRFLCQRPDGALYGSLHFDPEACSFRERLLEDGYNVYQSEAHGLPLHLPGNKSPHRDPAPRGPARFLPLPGLPPALPEPPGILAPQPPDVGSSDPLSMVGGSQGRSPSYAS

SEQ ID NO: 7 - protein, 31 aa, synthetic construct
  VARIANT 1..31: note = Description of artificial sequence: synthetic protein
  VARIANT 2: note = misc_feature - Xaa at position 2 is Gly or Val
  VARIANT 16: note = misc_feature - Xaa at position 16 is Glu or Gly
  VARIANT 27..28: note = misc_feature - Xaa at position 27 is Val or Lys. Xaa at position 28 is Lys or Asn
  VARIANT 30..31: note = misc_feature - Xaa at position 30 is Gly or Glu. Xaa at position 31 is Gly or Pro
HXEGTFTSDVSSYLEXQAAKEFIAWLXXGXX

SEQ ID NO: 8 - protein, 229 aa, synthetic construct
  VARIANT 1..229: note = Description of artificial sequence: synthetic protein
  VARIANT 15..17: note = misc_feature - Xaa at position 15 is Pro or Glu. Xaa at position 16 is Phe, Val or Ala. Xaa at position 17 is Leu, Glu or Ala
  VARIANT 79: note = misc_feature - Xaa at position 79 is Asn or Ala
  VARIANT 229: note = misc_feature - Xaa at position 229 is Lys or absent
ESKYGPPCPPCPAPXXXGGPSVFLFPPKPKDTLMISRTPEVTCVVVDVSQEDPEVQFNWYVDGVEVHNAKTKPREEQFXSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLGX

SEQ ID NO: 9 - protein, 181 aa, synthetic construct
  VARIANT 1..181: note = Description of artificial sequence: synthetic protein
  VARIANT 45: note = misc_feature - Xaa at position 45 is selected from Ala, Lys, Arg, Glu or Gln
  VARIANT 98: note = misc_feature - Xaa at position 98 is selected from Leu, Asp, Arg, Glu, Gln, Lys or Thr
  VARIANT 100: note = misc_feature - Xaa at position 100 is selected from Leu or Lys
  VARIANT 118: note = misc_feature - Xaa at position 118 is selected from Leu or Cys
  VARIANT 134: note = misc_feature - Xaa at position 134 is selected from Ala or Cys
  VARIANT 150..152: note = misc_feature - Xaa at position 150 is selected from Pro or Ala. Xaa at position 151 is selected from Gly or Ala. Xaa at position 152 is selected from Ile or Val
  VARIANT 167: note = misc_feature - Xaa at position 167 is selected from Ser, His, Cys or Arg
  VARIANT 170..172: note = misc_feature - Xaa at position 170 is selected from Gly, Glu, Ala, Asp or Ser at position 170. Xaa at position 171 is selected from Pro, Ala, Glu, Gln, Trp, Cys, Gly or Thr; Xaa at position 172 is selected from Ser, Leu, Val or Met
  VARIANT 175: note = misc_feature - Xaa at position 175 is selected from Arg, Leu, His or Pro
  VARIANT 179..180: note = misc_feature - Xaa at position 179 is selected from Tyr, Ser, Ala or Phe. Xaa at position 180 is selected from Ala, Ser, Glu or Gly
HPIPDSSPLLQFGGQVRQRYLYTDDAQQTEAHLEIREDGTVGGAXDQSPESLLQLKALKPGVIQILGVKTSRFLCQRPDGALYGSLHFDPEACSFREXLXEDGYNVYQSEAHGLPLHXPGNKSPHRDPAPRGPXRFLPLPGLPPALPEPXXXLAPQPPDVGSSDPLXMVXXXQGXSPSXXS

SEQ ID NO: 10 - protein, 31 aa, synthetic construct
  VARIANT 1..31: note = Description of artificial sequence: synthetic protein
  VARIANT 2: note = misc_feature - Xaa at position 2 is Gly or Val
  VARIANT 16: note = misc_feature - Xaa at position 16 is Glu or Gly
  VARIANT 30: note = misc_feature - Xaa at position 30 is Gly or GIu
HXEGTFTSDVSSYLEXQAAKEFIAWLVKGXG

SEQ ID NO: 11 - protein, 229 aa, synthetic construct
  VARIANT 1..229: note = Description of artificial sequence: synthetic protein
  VARIANT 16..17: note = misc_feature - Xaa at position 16 is Phe, Val or Ala. Xaa at position 17 is Leu, Glu or Ala
  VARIANT 229: note = misc_feature - Xaa at position 229 is Lys or absent
ESKYGPPCPPCPAPEXXGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLGX

SEQ ID NO: 12 - protein, 181 aa, synthetic construct
  VARIANT 1..181: note = Description of artificial sequence: synthetic protein
  VARIANT 98: note = misc_feature - Xaa at position 98 is selected from Leu, Asp, Arg or Glu
  VARIANT 167: note = misc_feature - Xaa at position 167 is selected from Ser, His, Cys or Arg
  VARIANT 171: note = misc_feature - Xaa at position 171 is selected from Ala or Gly
  VARIANT 175: note = misc_feature - Xaa at position 175 is selected from Arg, Leu, or Pro
  VARIANT 179: note = misc_feature - The Xaa at position 179 is selected from Tyr, Ser or Phe
HPIPDSSPLLQFGGQVRQRYLYTDDAQQTEAHLEIREDGTVGGAVDQSPESLLQLKALKPGVIQILGVKTSRFLCQRPDGALYGSLHFDPEACSFREXLLEDGYNVYQSEAHGLPLHLPGNKSPHRDPAPRGPARFLPLPGLPPALPEPPGILAPQPPDVGSSDPLXMVGXSQGXSPSXAS

SEQ ID NO: 13 - protein, 15 aa, synthetic construct
  REGION 1..15: note = Description of artificial sequence: synthetic protein
GGGGSGGGGSGGGGS

SEQ ID NO: 14 - protein, 31 aa, synthetic construct
  REGION 1..31: note = Description of artificial sequence: synthetic protein
HAEGTFTSDVSSYLEGQAAKEFIAWLVKGRG

SEQ ID NO: 15 - protein, 181 aa, synthetic construct
  REGION 1..181: note = Description of artificial sequence: synthetic protein
HPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPALPEPPGILAPQPPDVGSSDPLSMVGPSQGRSPSYAS

SEQ ID NO: 16 - protein, 470 aa, synthetic construct
  REGION 1..470: note = Description of artificial sequence: synthetic protein
HGEGTFTSDVSSYLEEQAAKEFIAWLVKGGGGGGGSGGGGSGGGGSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGSHPIPDSSPLLQFGGQVRQRYLYTDDAQQTEAHLEIREDGTVGGAADQSPESLLQLKALKPGVIQILGVKTSRFLCQRPDGALYGSLHFDPEACSFRERLLEDGYNVYQSEAHGLPLHLPGNKSPHRDPAPRGPARFLPLPGLPPALPEPPGILAPQPPDVGSSDPLSMVGGSQGRSPSYAS

SEQ ID NO: 17 - protein, 470 aa, synthetic construct
  REGION 1..470: note = Description of artificial sequence: synthetic protein
HGEGTFTSDVSSYLEEQAAKEFIAWLVKGGGGGGGSGGGGSGGGGSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLGGGGGSGGGGSGGGGSHPIPDSSPLLQFGGQVRQRYLYTDDAQQTEAHLEIREDGTVGGAADQSPESLLQLKALKPGVIQILGVKTSRFLCQRPDGALYGSLHFDPEACSFRERLLEDGYNVYQSEAHGLPLHLPGNKSPHRDPAPRGPARFLPLPGLPPALPEPPGILAPQPPDVGSSDPLSMVGGSQGRSPSFAS

SEQ ID NO: 18 - protein, 470 aa, synthetic construct
  REGION 1..470: note = Description of artificial sequence: synthetic protein
HGEGTFTSDVSSYLEEQAAKEFIAWLVKGGGGGGGSGGGGSGGGGSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLGGGGGSGGGGSGGGGSHPIPDSSPLLQFGGQVRQRYLYTDDAQQTEAHLEIREDGTVGGAADQSPESLLQLKALKPGVIQILGVKTSRFLCQRPDGALYGSLHFDPEACSFRERLLEDGYNVYQSEAHGLPLHLPGNKSPHRDPAPRGPARFLPLPGLPPALPEPPGILAPQPPDVGSSDPLSMVGASQGRSPSYAS

SEQ ID NO: 19 - protein, 470 aa, synthetic construct
  REGION 1..470: note = Description of artificial sequence: synthetic protein
HGEGTFTSDVSSYLEEQAAKEFIAWLVKGGGGGGGSGGGGSGGGGSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLGGGGGSGGGGSGGGGSHPIPDSSPLLQFGGQVRQRYLYTDDAQQTEAHLEIREDGTVGGAADQSPESLLQLKALKPGVIQILGVKTSRFLCQRPDGALYGSLHFDPEACSFRERLLEDGYNVYQSEAHGLPLHLPGNKSPHRDPAPRGPARFLPLPGLPPALPEPPGILAPQPPDVGSSDPLHMVGASQGRSPSYAS

SEQ ID NO: 20 - protein, 470 aa, synthetic construct
  REGION 1..470: note = Description of artificial sequence: synthetic protein
HGEGTFTSDVSSYLEEQAAKEFIAWLVKGGGGGGGSGGGGSGGGGSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLGGGGGSGGGGSGGGGSHPIPDSSPLLQFGGQVRQRYLYTDDAQQTEAHLEIREDGTVGGAADQSPESLLQLKALKPGVIQILGVKTSRFLCQRPDGALYGSLHFDPEACSFRERLLEDGYNVYQSEAHGLPLHLPGNKSPHRDPAPRGPARFLPLPGLPPALPEPPGILAPQPPDVGSSDPLHMVGASQGLSPSYAS

SEQ ID NO: 21 - protein, 470 aa, synthetic construct
  REGION 1..470: note = Description of artificial sequence: synthetic protein
HGEGTFTSDVSSYLEEQAAKEFIAWLVKGGGGGGGSGGGGSGGGGSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLGGGGGSGGGGSGGGGSHPIPDSSPLLQFGGQVRQRYLYTDDAQQTEAHLEIREDGTVGGAADQSPESLLQLKALKPGVIQILGVKTSRFLCQRPDGALYGSLHFDPEACSFRERLLEDGYNVYQSEAHGLPLHLPGNKSPHRDPAPRGPARFLPLPGLPPALPEPPGILAPQPPDVGSSDPLSMVEALQGRSPSYAS

SEQ ID NO: 22 - protein, 470 aa, synthetic construct
  REGION 1..470: note = Description of artificial sequence: synthetic protein
HGEGTFTSDVSSYLEEQAAKEFIAWLVKGGGGGGGSGGGGSGGGGSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLGGGGGSGGGGSGGGGSHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPALPEPPGILAPQPPDVGSSDPLSMVGPSQGRSPSYAS

SEQ ID NO: 23 - DNA, 1272 bp, synthetic construct
  misc_feature 1..1272: note = DNA-S1F1
  source 1..1272: mol_type = other DNA
gagtctaagtacggccctccctgccctccttgtcctgctcctgaagctgctggcggcccttccgtgttcctgttccccccaaagcccaaggacaccctgatgatctcccggacccccgaagtgacctgcgtggtggtggatgtgtcccaggaagatcccgaggtgcagttcaattggtacgtggacggcgtggaagtgcacaacgccaagaccaagcccagagaggaacagttcaactccacctaccgggtggtgtccgtgctgacagtgctgcaccaggactggctgaacggcaaagagtacaagtgcaaggtgtccaacaagggcctgcccagctccatcgaaaagaccatctccaaggccaagggccagccccgggaaccccaggtgtacacactgcctccaagccaggaagagatgaccaagaaccaggtgtccctgacctgtctcgtgaaaggcttctacccctccgatatcgccgtggaatgggagtccaacggccagcctgagaacaactacaagaccaccccccctgtgctggactccgacggctccttcttcctgtactccaagctgaccgtggacaagtccagatggcaggaaggcaacgtgttctcctgctccgtgatgcacgaggccctgcacaaccactacacccagaagtccctgtccctgtctctgggaggcggcggaggatctggcggaggtggaagcggaggcggtggatctcaccccatccccgacagcagccccctgctgcagttcggcggccaggtgaggcagaggtacctgtacaccgacgacgcccagcagaccgaggcccacctggagatcagggaggacggcaccgtgggcggcgccgccgaccagagccccgagagcctgctgcagctgaaggccctgaagcccggcgtgatccagatcctgggcgtgaagaccagcaggttcctgtgccagaggcccgacggcgccctgtacggcagcctgcacttcgaccccgaggcctgcagcttcagggagaggctgctggaggacggctacaacgtgtaccagagcgaggcccacggcctgcccctgcacctgcccggcaacaagagcccccacagggaccccgcccccaggggccccgccaggttcctgcccctgcccggcctgccccccgccctgcccgagccccccggcatcctggccccccagccccccgacgtgggcagcagcgaccccctgagcatggtgggaggatcccagggcaggagccccagctacgccagc

SEQ ID NO: 24 - DNA, 1410 bp, synthetic construct
  misc_feature 1..1410: note = DNA-D1F1
  source 1..1410: mol_type = other DNA
catggcgagggcacctttacctccgacgtgtcctcctacctggaagaacaggccgccaaagagtttatcgcctggctcgtgaagggcggtggtggcggcggaggatctggcggaggtggaagcggaggcggtggatctgagtctaagtacggccctccctgccctccttgtcctgctcctgaagctgctggcggcccttccgtgttcctgttccccccaaagcccaaggacaccctgatgatctcccggacccccgaagtgacctgcgtggtggtggatgtgtcccaggaagatcccgaggtgcagttcaattggtacgtggacggcgtggaagtgcacaacgccaagaccaagcccagagaggaacagttcaactccacctaccgggtggtgtccgtgctgacagtgctgcaccaggactggctgaacggcaaagagtacaagtgcaaggtgtccaacaagggcctgcccagctccatcgaaaagaccatctccaaggccaagggccagccccgggaaccccaggtgtacacactgcctccaagccaggaagagatgaccaagaaccaggtgtccctgacctgtctcgtgaaaggcttctacccctccgatatcgccgtggaatgggagtccaacggccagcctgagaacaactacaagaccaccccccctgtgctggactccgacggctccttcttcctgtactccaagctgaccgtggacaagtccagatggcaggaaggcaacgtgttctcctgctccgtgatgcacgaggccctgcacaaccactacacccagaagtccctgtccctgtctctgggaggcggcggaggatctggcggaggtggaagcggaggcggtggatctcaccccatccccgacagcagccccctgctgcagttcggcggccaggtgaggcagaggtacctgtacaccgacgacgcccagcagaccgaggcccacctggagatcagggaggacggcaccgtgggcggcgccgccgaccagagccccgagagcctgctgcagctgaaggccctgaagcccggcgtgatccagatcctgggcgtgaagaccagcaggttcctgtgccagaggcccgacggcgccctgtacggcagcctgcacttcgaccccgaggcctgcagcttcagggagaggctgctggaggacggctacaacgtgtaccagagcgaggcccacggcctgcccctgcacctgcccggcaacaagagcccccacagggaccccgcccccaggggccccgccaggttcctgcccctgcccggcctgccccccgccctgcccgagccccccggcatcctggccccccagccccccgacgtgggcagcagcgaccccctgagcatggtgggaggatcccagggcaggagccccagctacgccagc

SEQ ID NO: 25 - DNA, 1410 bp, synthetic construct
  misc_feature 1..1410: note = DNA-D1F2
  source 1..1410: mol_type = other DNA
catggcgagggcacctttacctccgacgtgtcctcctacctggaagaacaggccgccaaagagtttatcgcctggctcgtgaagggcggtggtggcggcggaggatctggcggaggtggaagcggaggcggtggatctgagtctaagtacggccctccctgccctccttgtcctgctcctgaagctgctggcggcccttccgtgttcctgttccccccaaagcccaaggacaccctgatgatctcccggacccccgaagtgacctgcgtggtggtggatgtgtcccaggaagatcccgaggtgcagttcaattggtacgtggacggcgtggaagtgcacaacgccaagaccaagcccagagaggaacagttcaactccacctaccgggtggtgtccgtgctgacagtgctgcaccaggactggctgaacggcaaagagtacaagtgcaaggtgtccaacaagggcctgcccagctccatcgaaaagaccatctccaaggccaagggccagccccgggaaccccaggtgtacacactgcctccaagccaggaagagatgaccaagaaccaggtgtccctgacctgtctcgtgaaaggcttctacccctccgatatcgccgtggaatgggagtccaacggccagcctgagaacaactacaagaccaccccccctgtgctggactccgacggctccttcttcctgtactccaggctgaccgtggacaagtccagatggcaggaaggcaacgtgttctcctgctccgtgatgcacgaggccctgcacaaccactacacccagaagtccctgtccctgtctctgggaggcggcggaggatctggcggaggtggaagcggaggcggtggatctcaccccatccccgacagcagccccctgctgcagttcggcggccaggtgaggcagaggtacctgtacaccgacgacgcccagcagaccgaggcccacctggagatcagggaggacggcaccgtgggcggcgccgccgaccagagccccgagagcctgctgcagctgaaggccctgaagcccggcgtgatccagatcctgggcgtgaagaccagcaggttcctgtgccagaggcccgacggcgccctgtacggcagcctgcacttcgaccccgaggcctgcagcttcagggagaggctgctggaggacggctacaacgtgtaccagagcgaggcccacggcctgcccctgcacctgcccggcaacaagagcccccacagggaccccgcccccaggggccccgccaggttcctgcccctgcccggcctgccccccgccctgcccgagccccccggcatcctggccccccagccccccgacgtgggcagcagcgaccccctgagcatggtgggaggatcccagggcaggagccccagctacgccagc

SEQ ID NO: 26 - DNA, 1410 bp, synthetic construct
  misc_feature 1..1410: note = DNA-D2F1
  source 1..1410: mol_type = other DNA
catggcgagggcacctttacctccgacgtgtcctcctacctggaagaacaggccgccaaagagtttatcgcctggctcgtgaagggcggtggtggcggcggaggatctggcggaggtggaagcggaggcggtggatctgagtctaagtacggccctccctgccctccttgtcctgctcctgaagctgctggcggcccttccgtgttcctgttccccccaaagcccaaggacaccctgatgatctcccggacccccgaagtgacctgcgtggtggtggatgtgtcccaggaagatcccgaggtgcagttcaattggtacgtggacggcgtggaagtgcacaacgccaagaccaagcccagagaggaacagttcaactccacctaccgggtggtgtccgtgctgacagtgctgcaccaggactggctgaacggcaaagagtacaagtgcaaggtgtccaacaagggcctgcccagctccatcgaaaagaccatctccaaggccaagggccagccccgggaaccccaggtgtacacactgcctccaagccaggaagagatgaccaagaaccaggtgtccctgacctgtctcgtgaaaggcttctacccctccgatatcgccgtggaatgggagtccaacggccagcctgagaacaactacaagaccaccccccctgtgctggactccgacggctccttcttcctgtactccaagctgaccgtggacaagtccagatggcaggaaggcaacgtgttctcctgctccgtgatgcacgaggccctgcacaaccactacacccagaagtccctgtccctgtctctgggaggcggcggaggatctggcggaggtggaagcggaggcggtggatctcaccccatccccgacagcagccccctgctgcagttcggcggccaggtgaggcagaggtacctgtacaccgacgacgcccagcagaccgaggcccacctggagatcagggaggacggcaccgtgggcggcgccgccgaccagagccccgagagcctgctgcagctgaaggccctgaagcccggcgtgatccagatcctgggcgtgaagaccagcaggttcctgtgccagaggcccgacggcgccctgtacggcagcctgcacttcgaccccgaggcctgcagcttcagggagaggctgctggaggacggctacaacgtgtaccagagcgaggcccacggcctgcccctgcacctgcccggcaacaagagcccccacagggaccccgcccccaggggccccgccaggttcctgcccctgcccggcctgccccccgccctgcccgagccccccggcatcctggccccccagccccccgacgtgggcagcagcgaccccctgagcatggtgggaggatcccagggcaggagccccagcttcgccagc

SEQ ID NO: 27 - DNA, 1410 bp, synthetic construct
  misc_feature 1..1410: note = DNA-D3F1
  source 1..1410: mol_type = other DNA
catggcgagggcacctttacctccgacgtgtcctcctacctggaagaacaggccgccaaagagtttatcgcctggctcgtgaagggcggtggtggcggcggaggatctggcggaggtggaagcggaggcggtggatctgagagcaagtacggccccccctgtcctccttgccccgcccctgaggccgccggcggccctagcgtgtttctgtttccccccaagcctaaagacaccctgatgatctccaggacccctgaggtgacctgtgtggtggtggacgtgagccaggaggaccccgaggtgcagttcaactggtacgtggatggcgtggaagtgcacaacgccaagaccaagcccagggaggagcaattcaacagcacctacagggtggtgagcgtcctcaccgtcctgcatcaggactggctgaacggcaaggagtacaagtgcaaagtgtccaacaagggcctgccttcctccatcgagaagaccatctccaaggctaagggccagcccagggaaccccaagtgtacaccctccccccctcccaggaggagatgaccaaaaaccaagtctccctgacctgcctggtgaagggcttctacccctccgatattgccgtcgagtgggagagcaacggccagcccgagaacaactataagaccaccccccccgtgctggattccgacggttcttttttcctgtatagcaagctgaccgtggacaagtccaggtggcaggagggcaacgtgttctcctgcagcgtgatgcacgaggccctccacaaccactacacccagaaatccctgtccctgtccctcggcggcggaggcggctccggcggcggcggcagcggaggcggaggaagccatcccattcccgactccagccccctgctgcagtttggcggccaagtgaggcagagatacctgtacaccgacgatgcccaacagacagaggctcacctggaaatcagggaggacggcaccgtgggcggagctgctgatcagagccccgagtccctcctccagctgaaggccctgaagcccggagtgatccagatcctgggcgtgaagacatccaggttcctgtgccagagacccgatggcgccctgtacggaagcctgcacttcgaccccgaggcttgctccttcagggagaggctgctggaggacggctacaacgtgtaccagtccgaggctcacggactccctctgcacctgcctggcaacaagagccctcacagagaccccgcccctagaggccctgctaggtttctgcccctgcctggcctgcctcctgctctgcccgagccccctggtattttagctcctcagcctcccgatgtgggaagcagcgaccccctgagcatggtgggagctagccagggcaggagccctagctacgccagc

SEQ ID NO: 28 - DNA, 1410 bp, synthetic construct
  misc_feature 1..1410: note = DNA-D4F1
  source 1..1410: mol_type = other DNA
catggcgagggcacctttacctccgacgtgtcctcctacctggaagaacaggccgccaaagagtttatcgcctggctcgtgaagggcggtggtggcggcggaggatctggcggaggtggaagcggaggcggtggatctgagagcaagtacggccccccctgtcctccttgccccgcccctgaggccgccggcggccctagcgtgtttctgtttccccccaagcctaaagacaccctgatgatctccaggacccctgaggtgacctgtgtggtggtggacgtgagccaggaggaccccgaggtgcagttcaactggtacgtggatggcgtggaagtgcacaacgccaagaccaagcccagggaggagcaattcaacagcacctacagggtggtgagcgtcctcaccgtcctgcatcaggactggctgaacggcaaggagtacaagtgcaaagtgtccaacaagggcctgccttcctccatcgagaagaccatctccaaggctaagggccagcccagggaaccccaagtgtacaccctccccccctcccaggaggagatgaccaaaaaccaagtctccctgacctgcctggtgaagggcttctacccctccgatattgccgtcgagtgggagagcaacggccagcccgagaacaactataagaccaccccccccgtgctggattccgacggttcttttttcctgtatagcaagctgaccgtggacaagtccaggtggcaggagggcaacgtgttctcctgcagcgtgatgcacgaggccctccacaaccactacacccagaaatccctgtccctgtccctcggcggcggaggcggctccggcggcggcggcagcggaggcggaggaagccatcccattcccgactccagccccctgctgcagtttggcggccaagtgaggcagagatacctgtacaccgacgatgcccaacagacagaggctcacctggaaatcagggaggacggcaccgtgggcggagctgctgatcagagccccgagtccctcctccagctgaaggccctgaagcccggagtgatccagatcctgggcgtgaagacatccaggttcctgtgccagagacccgatggcgccctgtacggaagcctgcacttcgaccccgaggcttgctccttcagggagaggctgctggaggacggctacaacgtgtaccagtccgaggctcacggactccctctgcacctgcctggcaacaagagccctcacagagaccccgcccctagaggccctgctaggtttctgcccctgcctggcctgcctcctgctctgcccgagccccctggtattttagctcctcagcctcccgatgtgggaagcagcgaccccctgcacatggtgggagctagccagggcaggagccctagctacgccagc

SEQ ID NO: 29 - DNA, 1410 bp, synthetic construct
  misc_feature 1..1410: note = DNA-D5F1
  source 1..1410: mol_type = other DNA
catggcgagggcacctttacctccgacgtgtcctcctacctggaagaacaggccgccaaagagtttatcgcctggctcgtgaagggcggtggtggcggcggaggatctggcggaggtggaagcggaggcggtggatctgagagcaagtacggccccccctgtcctccttgccccgcccctgaggccgccggcggccctagcgtgtttctgtttccccccaagcctaaagacaccctgatgatctccaggacccctgaggtgacctgtgtggtggtggacgtgagccaggaggaccccgaggtgcagttcaactggtacgtggatggcgtggaagtgcacaacgccaagaccaagcccagggaggagcaattcaacagcacctacagggtggtgagcgtcctcaccgtcctgcatcaggactggctgaacggcaaggagtacaagtgcaaagtgtccaacaagggcctgccttcctccatcgagaagaccatctccaaggctaagggccagcccagggaaccccaagtgtacaccctccccccctcccaggaggagatgaccaaaaaccaagtctccctgacctgcctggtgaagggcttctacccctccgatattgccgtcgagtgggagagcaacggccagcccgagaacaactataagaccaccccccccgtgctggattccgacggttcttttttcctgtatagcaagctgaccgtggacaagtccaggtggcaggagggcaacgtgttctcctgcagcgtgatgcacgaggccctccacaaccactacacccagaaatccctgtccctgtccctcggcggcggaggcggctccggcggcggcggcagcggaggcggaggaagccatcccattcccgactccagccccctgctgcagtttggcggccaagtgaggcagagatacctgtacaccgacgatgcccaacagacagaggctcacctggaaatcagggaggacggcaccgtgggcggagctgctgatcagagccccgagtccctcctccagctgaaggccctgaagcccggagtgatccagatcctgggcgtgaagacatccaggttcctgtgccagagacccgatggcgccctgtacggaagcctgcacttcgaccccgaggcttgctccttcagggagaggctgctggaggacggctacaacgtgtaccagtccgaggctcacggactccctctgcacctgcctggcaacaagagccctcacagagaccccgcccctagaggccctgctaggtttctgcccctgcctggcctgcctcctgctctgcccgagccccctggtattttagctcctcagcctcccgatgtgggaagcagcgaccccctgcacatggtgggagctagccagggcctgagccctagctacgccagc

SEQ ID NO: 30 - DNA, 1410 bp, synthetic construct
  misc_feature 1..1410: note = DNA-D6F1
  source 1..1410: mol_type = other DNA
catggcgagggcacctttacctccgacgtgtcctcctacctggaagaacaggccgccaaagagtttatcgcctggctcgtgaagggcggtggtggcggcggaggatctggcggaggtggaagcggaggcggtggatctgagtctaagtacggccctccctgccctccttgtcctgctcctgaagctgctggcggcccttccgtgttcctgttccccccaaagcccaaggacaccctgatgatctcccggacccccgaagtgacctgcgtggtggtggatgtgtcccaggaagatcccgaggtgcagttcaattggtacgtggacggcgtggaagtgcacaacgccaagaccaagcccagagaggaacagttcaactccacctaccgggtggtgtccgtgctgacagtgctgcaccaggactggctgaacggcaaagagtacaagtgcaaggtgtccaacaagggcctgcccagctccatcgaaaagaccatctccaaggccaagggccagccccgggaaccccaggtgtacacactgcctccaagccaggaagagatgaccaagaaccaggtgtccctgacctgtctcgtgaaaggcttctacccctccgatatcgccgtggaatgggagtccaacggccagcctgagaacaactacaagaccaccccccctgtgctggactccgacggctccttcttcctgtactccaagctgaccgtggacaagtccagatggcaggaaggcaacgtgttctcctgctccgtgatgcacgaggccctgcacaaccactacacccagaagtccctgtccctgtctctgggaggcggcggaggatctggcggaggtggaagcggaggcggtggatctcaccccatccccgacagcagccccctgctgcagttcggcggccaggtgaggcagaggtacctgtacaccgacgacgcccagcagaccgaggcccacctggagatcagggaggacggcaccgtgggcggcgccgccgaccagagccccgagagcctgctgcagctgaaggccctgaagcccggcgtgatccagatcctgggcgtgaagaccagcaggttcctgtgccagaggcccgacggcgccctgtacggcagcctgcacttcgaccccgaggcctgcagcttcagggagaggctgctggaggacggctacaacgtgtaccagagcgaggcccacggcctgcccctgcacctgcccggcaacaagagcccccacagggaccccgcccccaggggccccgccaggttcctgcccctgcccggcctgccccccgccctgcccgagccccccggcatcctggccccccagccccccgacgtgggcagcagcgaccccctgagcatggtggaggccctgcagggcaggagccccagctacgccagc

SEQ ID NO: 31 - DNA, 1410 bp, synthetic construct
  misc_feature 1..1410: note = DNA-D7F1
  source 1..1410: mol_type = other DNA
catggcgagggcacctttacctccgacgtgtcctcctacctggaagaacaggccgccaaagagtttatcgcctggctcgtgaagggcggtggtggcggcggaggatctggcggaggtggaagcggaggcggtggatctgagtctaagtacggccctccctgccctccttgtcctgctcctgaagctgctggcggcccttccgtgttcctgttccccccaaagcccaaggacaccctgatgatctcccggacccccgaagtgacctgcgtggtggtggatgtgtcccaggaagatcccgaggtgcagttcaattggtacgtggacggcgtggaagtgcacaacgccaagaccaagcccagagaggaacagttcaactccacctaccgggtggtgtccgtgctgacagtgctgcaccaggactggctgaacggcaaagagtacaagtgcaaggtgtccaacaagggcctgcccagctccatcgaaaagaccatctccaaggccaagggccagccccgggaaccccaggtgtacacactgcctccaagccaggaagagatgaccaagaaccaggtgtccctgacctgtctcgtgaaaggcttctacccctccgatatcgccgtggaatgggagtccaacggccagcctgagaacaactacaagaccaccccccctgtgctggactccgacggctccttcttcctgtactccaagctgaccgtggacaagtccagatggcaggaaggcaacgtgttctcctgctccgtgatgcacgaggccctgcacaaccactacacccagaagtccctgtccctgtctctgggaggcggcggaggatctggcggaggtggaagcggaggcggtggatctcaccccatccccgacagcagccccctgctgcagttcggcggccaggtgaggcagaggtacctgtacaccgacgacgcccagcagaccgaggcccacctggagatcagggaggacggcaccgtgggcggcgccgccgaccagagccccgagagcctgctgcagctgaaggccctgaagcccggcgtgatccagatcctgggcgtgaagaccagcaggttcctgtgccagaggcccgacggcgccctgtacggcagcctgcacttcgaccccgaggcctgcagcttcagggagctgctgctggaggacggctacaacgtgtaccagagcgaggcccacggcctgcccctgcacctgcccggcaacaagagcccccacagggaccccgcccccaggggccccgccaggttcctgcccctgcccggcctgccccccgccctgcccgagccccccggcatcctggccccccagccccccgacgtgggcagcagcgaccccctgagcatggtgggaccttcccagggcaggagccccagctacgccagc

SEQ ID NO: 32 - protein, 10 aa, synthetic construct
  REGION 1..10: note = Description of artificial sequence: synthetic protein
GGGGSGGGGS

SEQ ID NO: 33 - protein, 16 aa, synthetic construct
  REGION 1..16: note = Description of artificial sequence: synthetic protein
GGGGSGGGGSGGGGSA

SEQ ID NO: 34 - protein, 15 aa, synthetic construct
  REGION 1..15: note = Description of artificial sequence: synthetic protein
GGGGGSGGGSGGGGS

SEQ ID NO: 35 - protein, 8 aa, synthetic construct
  REGION 1..8: note = Description of artificial sequence: synthetic protein
GGGKGGGG

SEQ ID NO: 36 - protein, 8 aa, synthetic construct
  REGION 1..8: note = Description of artificial sequence: synthetic protein
GGGNGSGG

SEQ ID NO: 37 - protein, 8 aa, synthetic construct
  REGION 1..8: note = Description of artificial sequence: synthetic protein
GGGCGGGG

SEQ ID NO: 38 - protein, 5 aa, synthetic construct
  REGION 1..5: note = Description of artificial sequence: synthetic protein
GPNGG

(19) United States
(12) Patent Application Publication
(10) Pub. No.: US 2025/0084144 A1
(43) Pub. Date: Mar. 13, 2025

(54) DUAL-TARGET FUSION PROTEINS COMPRISING FGF21 AND GLP-1, CONNECTED THROUGH A FC PORTION OF AN IMMUNOGLOBULIN

(71) Applicant: SUNSHINE LAKE PHARMA CO., LTD., Dongguan (CN)

(72) Inventors: Chao CHEN, DONGGUAN (CN); Shushan LIN, DONGGUAN (CN); Yu LI, DONGGUAN (CN); Xiaofeng CHEN, DONGGUAN (CN); Liang LIU, DONGGUAN (CN); Zheng FU, DONGGUAN (CN)

(73) Assignee: SUNSHINE LAKE PHARMA CO., LTD., Dongguan (CN)

(21) Appl. No.: 18/805,686

(22) Filed: Aug. 15, 2024

Related U.S. Application Data
(63) Continuation of application No. 16/485,153, filed on Aug. 10, 2019, now Pat. No. 12,145,974.

(30) Foreign Application Priority Data
Mar. 14, 2017 (CN) 201710148158.7
Feb. 8, 2018 (CN) 201810129427.X

(51) Int. Cl. C07K 14/605; A61P 3/04; C07K 14/50

(52) U.S. Cl. CPC C07K 14/605; A61P 3/04; C07K 14/50; C07K 2319/30

(57) ABSTRACT

Provided is a fusion protein containing GLP-1 and FGF21 linked through an immunoglobulin Fc portion. Each of the GLP-1 and FGF21 can be substituted with its analogues or variants and, the Fc portion, which can be derived from IgG4, can include substitutions that further enhance the fusion protein's stability and activity. These fragments can be linked together through peptide linkers. The fusion protein can be used clinically to reduce glucose and lipid levels and body weight.

### CROSS REFERENCE TO RELATED APPLICATIONS

This is a continuation application of U.S. application Ser. No. 16/485,153, filed Aug. 10, 2019, which is a U.S. national stage application of the International Patent Application No. PCT/CN2018/078925, filed Mar. 14, 2018, which claims priority and benefits of Chinese Patent Application No. 201710148158.7, filed with the State Intellectual Property Office on Mar. 14, 2017 and Chinese Patent Application No. 201810129427.X, filed with the State Intellectual Property Office on Feb. 8, 2018, all of which are incorporated herein by reference in their entirety.

### FIELD OF THE INVENTION

The present invention relates to fusion proteins comprising fibroblast growth factor 21 (FGF21) and glucagon-like peptide-1 (GLP-1), connected through an immunoglobulin Fc portion, and uses of the fusion proteins to treat metabolic diseases.

### BACKGROUND OF THE INVENTION

At present, there are three categories of drugs for the treatment of diabetes: oral small molecule drugs, insulin and GLP-1 receptor agonist drugs. Long-term treatment of small molecule drugs leads to obvious side effects, and post-diabetic glycemic control is not satisfactory. Treatment of diabetes with insulin requires multiple injections (at least once daily), and hypoglycemia is easily triggered by differences in individual dosages. A single GLP-1 receptor agonist is not a first-line agent and has limited efficacy in cardiovascular complications caused by abnormal metabolism of diabetes.

Glucagon-like peptide-1 (GLP-1) is a type of incretin secreted by intestinal L cells. It stimulates islet β-cells to secrete insulin, maintaining insulin balance in patients. Natural GLP-1 survives only up to 2 minutes in vitro and therefore is not effective as a drug. For a few years now, several parties including Eli Lilly, Novo Nordisk, GSK have been competitively seeking to transform the protein in order to obtain a long-acting GLP-1 class of hypoglycemic drugs. Currently, the longest-acting of GLP-1 drugs, such as dulaglutide, can be administered once per week by fusing an antibody fragment to the GLP-1 protein. However, studies find that the glucose-lowering curve of dulaglutide is not stable, and blood glucose begins to rise on the day after treatment. Thus, this situation is not ideal for controlling diabetes, and it may lead to a variety of complications.

Type 2 diabetes has two major hallmarks: peripheral insulin resistance and impaired glucose-dependent insulin secretion of pancreatic beta cells. Metabolic disorders such as diabetes are often caused by a variety of complex factors, so the treatment of multiple metabolic pathways is considered much more advantageous.

Fibroblast growth factor 21 (FGF21) is a recently discovered cytokine. It can stimulate GLUT1 receptors and promote glucose transport into cells, so it may be used as a drug to treat diabetes. However, FGF21 faces a huge problem when it comes to medicinal properties. FGF21 has a short half-life, about one hour in a mouse model (but maximum bioactivity can be maintained for more than six hours). The reason FGF21 rapidly degrades is because of proteases in the body and its propensity to form aggregates in vitro resulting in immunogenicity that is detrimental to half-life extension.

In recent years, various attempts have been made to increase the stability of FGF21 in vivo in order to develop the pharmaceutical use of FGF21, such as mutating at different sites, inserting amino acids/fatty chains, or deleting some amino acids which were made on the basis of FGF21 native sequences, such as those published in WO 2003011213, WO 2005072769, WO 2005061712, WO 2006065582, WO 2010065439, WO2013052311, WO 2013188182, WO 2013033452, WO 2005091944, WO 2012066075, WO 2013188181.

### SUMMARY

The present disclosure provides a dual-target fusion protein comprising GLP-1 and FGF21, with differing mutation sites from previously disclosed fusion proteins. The inventors have surprisingly found that the dual-target fusion protein of the present invention has better stability and significantly prolonged half-life, and exhibits synergistic effects in reducing blood sugar, lipid, weight, and so on.

In one aspect of the present invention, there is provided a fusion protein, which comprises or consists of a first polypeptide: GLP-1 or an analogue thereof, a second polypeptide: an IgG4 Fc portion and a third polypeptide: FGF21 or a variant thereof, wherein the first polypeptide is linked to the second polypeptide via a first linker and the third polypeptide is linked to the second polypeptide via a second linker, and wherein serine at position 228 of the IgG4 Fc portion is replaced by proline and arginine at position 409 of the IgG4 Fc portion is replaced by lysine, as compared to a wild-type IgG4 Fc portion. Therefore, the safety and stability of the fusion protein is increased, leading to a significantly prolonged half-life.

The first polypeptide, the GLP-1 analogue, comprises the native GLP-1 sequence (SEQ ID NO: 14) having 1 to 10 (such as 1, 2, 3, 4, 5, 6, 7, 8, 9 or 10) amino acid substitutions, deletions or insertions, thereby prolonging the half-life of GLP-1 in vivo, while retaining or enhancing the biological activity of GLP-1.

In some embodiments, the GLP-1 analogue comprises or consists of an amino acid sequence of SEQ ID NO: 7 (HX1EGTFTSDV SSYLEX2QAAK EFIAWLX3X4GX5 X6), wherein X1 is selected from G or V; X2 is selected from E or G; X3 is selected from V or K; X4 is selected from K or N; X5 is selected from G or E; X6 is selected from G or P or absent.

In other embodiments, the GLP-1 analogue comprises or consists of an amino acid sequence of SEQ ID NO: 1.

The second polypeptide, in some embodiments, the IgG4 Fc portion further comprises any one or more mutations selected from the following: E233P, F234V or F234A, L235E or L235A, N297A, K447 as compared to a wild-type IgG4 Fc portion.

In other embodiments, the IgG4 Fc portion comprises any one of the following combination of mutations:
1) S228P and R409K;
2) S228P, E233P and R409K;
3) S228P, N297A and R409K;
4) S228P, F234A, L235A and R409K;
5) S228P, F234V, L235E and R409K; and
6) S228P, F234A, L235A, R409K and K447, as compared to a wild-type IgG4 Fc portion.

In still other embodiments, the IgG4 Fc portion comprises or consists of an amino acid sequence of SEQ ID NO: 2.

The third polypeptide, the FGF21 variant, comprises the native FGF21 sequence having one or more amino acid substitutions, deletions or insertions, thereby prolonging the in vivo half-life of FGF21 and preserving or enhancing the biological activity of FGF21.

In some embodiments, the FGF21 variant comprises at least one amino acid mutation at position 170 or position 171 or position 172; the amino acid mutation at position 170 is selected from the group consisting of G170E, G170A, G170C, G170D, G170N, and G170S; the amino acid mutation at position 171 is selected from the group consisting of P171A, P171E, P171H, P171Q, P17IT, P171Y, P171W, P171C, P171G, P171S, and P171T; the amino acid mutation at position 172 is selected from the group consisting of S172L, S1721, S172V, S172A, and S172M. In some embodiments, the amino acid mutation at position 170 is selected from the group consisting of G170E, G170A, G170D, and G170S; the amino acid mutation at position 171 is selected from the group consisting of P171A, P171E, P171Q, P171W, P171C, P171G, and P171T; the amino acid mutation at position 172 is selected from the group consisting of S172L, S172V, and S172M. In some embodiments, the amino acid mutation at position 170 is selected from the group consisting of G170E, and G170A; the amino acid mutation at position 171 is selected from the group consisting of P171A, P171C, and P171G; the amino acid mutation at position 172 is S172L.

In some embodiments, the FGF21 variant further comprises one or more mutations at one or more sites selected from the group consisting of 45, 98, 100, 118, 134, 150, 151, 152, 167, 175, 179, and 180. In some embodiments, the amino acid mutation at position 45 is selected from the group consisting of A45K, A45R, A45E, and A45Q; the amino acid mutation at position 98 is selected from the group consisting of L98D, L98R, L98E, L98Q, L98K, and L98T; the amino acid mutation at position 100 is L100K; the amino acid mutation at position 118 is L118C; the amino acid at position 134 is A134C; the amino acid mutation at position 150 is P150A; the amino acid mutation at position 151 is G151A; the amino acid at position 152 is I152V; the amino acid mutation at position 167 is selected from the group consisting of S167H, S167C, and S167R; the amino acid mutation at position 175 is selected from the group consisting of R175L, R175H, and R175P; the amino acid at position 179 is selected from the group consisting of Y179S, Y179A, and Y179F; the amino acid mutation at position 180 is selected from the group consisting of A180S, A180E, and A180G.

In some embodiments, the FGF21 variant further comprises one or more mutations at one or more sites selected from the group consisting of 45, 98, 100, 167, 175, 179, and 180. In some embodiments, the amino acid mutation at position 45 is selected from the group consisting of A45K, A45R, A45E, and A45Q; the amino acid mutation at position 98 is selected from the group consisting of L98D, L98R, L98E, L98Q, L98K, and L98T; the amino acid mutation at position 100 is L100K; the amino acid mutation at position 167 is selected from the group consisting of S167H, and S167C; the amino acid mutation at position 175 is selected from the group consisting of R175L, and R175H; the amino acid at position 179 is selected from the group consisting of Y179S, Y179A, and Y179F; the amino acid mutation at position 180 is selected from the group consisting of A180S, A180E, and A180G.

In other embodiments, the FGF21 variant further comprises one or more mutations at one or more sites selected from the group consisting of 45, 98, 167, 175, 179 and 180. In some embodiments, the amino acid mutation at position 45 is selected from the group consisting of A45K, A45R, A45E, and A45Q; the amino acid mutation at position 98 is selected from an the group consisting of L98D, L98R, L98E, L98Q, L98K, and L98T; the amino acid mutation at position 167 is selected from the group consisting of S167H, and S167C; the amino acid mutation at position 175 is selected from the group consisting of R175L, and R175H; the amino acid at position 179 is selected from the group consisting of Y179S, Y179A, and Y179F; the amino acid mutation at position 180 is selected from the group consisting of A180S, A180E, and A180G.

In other embodiments, the FGF21 variant further comprises one or more mutations at one or more sites selected from the group consisting of 98, 167, 175, and 179. In some embodiments, the amino acid mutation at position 98 is selected from the group consisting of L98D, L98R, L98E, and L98K; the amino acid mutation at position 167 is selected from the group consisting of S167H, and S167C; the amino acid at position 175 is selected from the group consisting of: R175L, R175H; the amino acid mutation at position 179 is selected from the group consisting of Y179S, and Y179F.

In other embodiments, the FGF21 variant further comprises one or more mutations at one or more sites selected from the group consisting of 98, 167, 175, and 179. In some embodiments, the amino acid mutation at position 98 is selected from the group consisting of L98D, and L98R; the amino acid mutation at position 167 is S167H; the amino acid at position 175 is R175L; the amino acid mutation at position 179 is Y179F.

In some embodiments, the FGF21 variant comprises at least amino acid mutations at one or more sites of 98 or 171, wherein the amino acid mutation at position 98 is selected from the group consisting of L98D, and L98R; the amino acid mutation at position 170 is selected from the group consisting of P171A, and P171G. In some embodiments, the FGF21 variant further comprises one or more mutations at one or more sites selected from the group consisting of 167, 175, and 179, wherein the amino acid mutation at position 167 is selected from the group consisting of S167H, and S167C; the amino acid mutation at position 175 is selected from the group consisting of R175L, R175H, and R175P; the amino acid at position 179 is selected from the group consisting of Y179S, and Y179F.

In some embodiments, the FGF21 variant comprises at least amino acid mutations at one or more sites of 98 or 171, wherein, the amino acid mutation at position 98 is L98R; the amino acid mutation at position 170 is selected from the group consisting of P171A, and P171G. In some embodiments, the FGF21 variant further comprises one or more mutations at one or more sites selected from the group consisting of 167, 175, and 179, wherein the amino acid mutation at position 167 is S167H; the amino acid mutation at position 175 is R175L; the amino acid at position 179 is Y179F.

In other embodiments, the FGF21 variant comprises any one of the following combination of mutations:
1) L98R and P171G;
2) L98R and P171A;
3) L98R, P171G and Y179F;
4) L98R, P171G and A180E;
5) L98R, G170E, P171A and S172L;
6) L98R, S167H and P171A; and
7) L98R, S167H, P171A and R175L.

In yet other embodiments, the FGF21 variant further comprises:
a) amino-terminal truncation of not more than 8 amino acid residues;
b) carboxy-terminal truncation of not more than 12 amino acid residues;
c) amino-terminal truncation of not more than 8 amino acid residues and carboxy-terminal truncation of not more than 12 amino acid residues; or
d) carboxy-terminal extension of no more than 6 amino acid residues.

In the present invention, the first polypeptide and the third polypeptide are respectively fused with the IgG4 Fc portion through a first linker and a second linker, and the first linker and/or the second linker comprise a polypeptide of 5 to 50 amino acid residues, and the polypeptide comprises at least 50% G. In some embodiments, the first linker and the second linker each independently comprise 1, 2 or 3 G-rich polypeptides. In other embodiments, the first linker and the second linker are each independently selected from GGGGSGGGGS (SEQ ID NO: 32) or GGGGSGGGGSGGGGS (SEQ ID NO: 13) or GGGGSGGGGSGGGGSA (SEQ ID NO: 33).

In some embodiments, the fusion protein is a homodimeric fusion protein.

In some embodiments, the fusion protein is a non-homodimeric fusion protein.

In another aspect of the invention, polynucleotides are provided that encode the fusion proteins of the invention.

In a further aspect of the invention, there is also provided a vector comprising a polynucleotide of the invention.

In a further aspect of the invention, there is also provided a host cell comprising a vector of the invention.

In a further aspect of the invention, there is also provided a method of preparing a fusion protein of the invention, wherein the method comprises expressing a vector of the invention in a host cell.

In another aspect of the present invention, there is also provided a composition (such as a pharmaceutical composition) comprising the fusion protein of the present invention, and optionally one or more pharmaceutically acceptable adjuvants. Such pharmaceutically acceptable adjuvants include, but are not limited to, pharmaceutically acceptable carriers, excipients, diluents, vehicles, and other adjuvants required in pharmaceutical preparations, or any combination thereof.

The fusion proteins or compositions (e.g., pharmaceutical compositions) of the present invention can be used for the treatment of metabolic diseases. The metabolic diseases may be selected from diabetes, obesity and hepatic steatosis.

In another aspect of the present invention, there is also provided use of the fusion protein of the present invention in the preparation of a pharmaceutical composition. In some embodiments, the pharmaceutical composition is used for the treatment of metabolic diseases. For example, the metabolic diseases may be selected from diabetes, obesity and hepatic steatosis.

In another aspect of the present invention, there is also provided a method of treating metabolic diseases, which comprises administering a therapeutically effective amount of the fusion protein or pharmaceutical composition of the present invention to a subject in need. For example, the metabolic diseases may be selected from diabetes, obesity and hepatic steatosis.

In another aspect of the present invention, there is provided a fusion protein or pharmaceutical composition for the treatment of metabolic diseases. The metabolic disease may be selected from diabetes, obesity and hepatic steatosis.

The fusion protein provided herein has the following advantages: the fusion protein has improved stability and it is safer due to avoiding the dissociation of the dimer and thus avoiding the random combination with the antibody in the body; the fusion protein has a longer half-life, the administration period is greater than one week, and patient compliance is increased; it does not trigger ADCC or CDC effect. In another aspect, the homodimeric fusion protein comprising the first polypeptide, the second polypeptide and the third polypeptide provided herein has unique advantages including a good biological activity in metabolic diseases, a stable hypoglycemic effect because of the unique synergistic effect exerted by the first polypeptide and the third polypeptide, and other biological activities such as good hypolipaemic, weight loss, etc.

### DESCRIPTION OF THE DRAWING

FIG. 1 shows a schematic diagram of a homodimeric fusion protein in one embodiment.

FIG. 2 shows plots of cAMP concentration as a function of dulaglutide or D3F1 detected by using a GLP-IR cell model.

FIG. 3 shows microcalorimetric differential scanning calorimetry curves of fusion proteins D1F1, D1F2, D6F1 and D7F1.

FIG. 4 shows the blood glucose levels after administration of dulaglutide, S1F1 and D1F1 in db/db mouse model.

FIG. 5 shows the blood glucose levels after administration of fusion proteins D1F1, D2F1 and D7F1 in db/db mouse mode.

FIG. 6 shows the hypoglycemic effects after administration of fusion proteins D3F1, D4F1 and D5F1 in db/db mouse model.

FIG. 7 shows the reduced total cholesterol effects after administration of dulaglutide, S1F1 and D1F1 in db/db mouse model.

FIG. 8 shows the reduced triglyceride effects after administration of dulaglutide, S1F1 and D1F1 in db/db mouse model.

FIG. 9 shows the reduced triglyceride effects after administration of fusion proteins D1F2, D2F1 and D7F1 in db/db mouse model.

FIG. 10 shows the reduced total cholesterol effects after administration of fusion proteins D1F2, D2F1 and D7F1 in db/db mouse model.

FIG. 11 shows the reduced high density lipoprotein effects after administration of fusion proteins D1F1, D2F1 and D7F1 in db/db mouse model.

FIG. 12 shows the reduced low density lipoprotein effects after administration of fusion proteins D1F1, D2F1 and D7F1 in db/db mouse model.

FIG. 13 shows the reduced triglyceride effects after administration of fusion proteins D3F1, D4F1, D5F1 in db/db mouse model.

FIG. 14 shows the reduced total cholesterol effects after administration of fusion proteins D3F1, D4F1 and D5F1 in db/db mouse model.

FIG. 15 shows the reduced low density lipoprotein effects after administration of fusion proteins D3F1, D4F1 and D5F1 in db/db mouse model.

FIG. 16 shows the reduced the weight effects after administration of dulaglutide, S1F1 and D1F1 in db/db mouse model.

FIG. 17 shows the hypoglycemic effects after administration of dulaglutide and D5F1 in ob/ob mouse model.

FIG. 18 shows the blood glucose concentration after administration of dulaglutide and D5F1 after 16 hours of fasting in the ob/ob mouse model.

FIG. 19 shows the improving hepatic steatosis effects after administration of dulaglutide and D5F1 in the ob/ob mouse model.

### EXAMPLES

Other features and advantages of the present invention will be apparent from the following detailed description. However, it should be understood that the detailed description and specific examples, while indicating preferred embodiments of the invention, are given by way of illustration only, and in that many changes and modifications within the spirit and scope of the invention will become apparent to those skilled in the art from the detailed description.

### DEFINITION OF TERMINOLOGY

As used herein, “native GLP-1 sequence” refers to a native GLP-1 (7-37) sequence with biological activity that is derived from a naturally occurring GLP-1 sequence by excision of an N-terminal 6 peptide. The native GLP-1 sequence, i.e. the native GLP-1 (7-37) sequence, is: HAEGTFTSDV SSYLEGQAAK EFIAWLVKGR G (SEQ ID NO: 14).

As used herein, “biological activity of GLP-1” refers to a variety of biological effects induced by GLP-1, such as stimulating insulin secretion, inhibiting glucagon secretion, inhibiting gastric emptying, inhibiting gastric or intestinal motility and inducing weight loss. A striking feature of GLP-1 is that it can stimulate insulin secretion without risks associated with hypoglycemia.

The “Fc portion” described herein consists of a hinge region of an antibody, CH2 and CH3 constant region structures. The antibody comprises two functionally independent moieties, a variable domain called “Fab” that binds to an antigen, and a constant domain called “Fc” that is involved in effector functions (e.g., complement activation and attack by phagocytes). Fc has a long serum half-life, whereas Fab is short-lived (Capon et al., 1989, Nature 337:525-31). When linked to a therapeutic protein, the Fc domain may provide longer half-lives or incorporate such functions as Fc receptor binding, protein A binding, complement fixation or even placental transfer (Capon et al., 1989). The term “Fc portion” as used herein refers to wild-type Fc portion sequences derived from natural antibodies (e.g., human IgG1, IgG2, IgG3, or IgG4) and also encompasses their variants. The variants may include one or more amino acid substitutions, additions and/or deletions such as those disclosed herein. In some embodiments, the Fc portion variants maintain the activity of the wild-type Fc portion, such as binding to an Fc receptor.

The amino acid numbers of the IgG4 Fc portion described herein are numbered according to the EU numbering system, for example, “S228P” means that serine at position 228 according to the EU numbering system is replaced by proline; “K447” means that lysine at position 447 according to the EU numbering system is deleted or absent.

The FGF21 wild-type sequence contains 209 amino acids with the NCBI reference sequence number NP_061986.1; a mature FGF21 sequence contains 181 amino acids, and lacks a leader sequence that the FGF21 wild-type contains. As used herein, a “native FGF21 sequence” refers to the mature FGF21 sequence with the following amino acid sequence (SEQ ID NO: 15), the “FGF21 variant” described herein is a mutant polypeptide having the following FGF21 amino acid sequence (SEQ ID NO: 15), the amino acid mutation site numbers are numbered in the following sequence (SEQ ID NO: 15). The amino acid sequence of the FGF21 variant differs from the native FGF21 amino acid sequence by one or more amino acids. FGF21 variants can be obtained by modification with natural or non-naturally occurring amino acids at particular positions in the native FGF21 polypeptide, such modifications include insertion, replacement or deletion of one or more conservative or non-conservative amino acids at a particular position, as well as introduction of non-amino acid structures at specific positions.

SEQ ID NO: 15: HPIPDSSPLL QFGGQVRQRY LYTDDAQQTE AHLEIREDGT VGGAADQSPE SLLQLKALKP GVIQILGVKT SRFLCQRPDG ALYGSLHFDP EACSFRELLL EDGYNVYQSE AHGLPLHLPG NKSPHRDPAP RGPARFLPLP GLPPALPEPP GILAPQPPDV GSSDPLSMVG PSQGRSPSYA S

FGF-21 variants may additionally or alternatively comprise deletion of amino acids, which may be N-terminal truncations, C-terminal truncations or internal deletions or any combination thereof. These variants comprising N-terminal truncation, C-terminal truncation and/or internal deletion are referred to as “deletion variants” or “fragments” in the context of the present invention. The terms “deletion variants” and “fragments” are used interchangeably herein. Fragments may be naturally occurring (e.g., splice variants) or artificially constructed, e.g., through genetic means.

“Conservative amino acid substitutions” may include residues of a native amino acid residue (i.e., a residue present at a given position in the wild-type FGF21 polypeptide sequence) replaced by a non-native residue (i.e., a residue not present at a given position in the wild-type FGF21 polypeptide sequence), so that there is little or no effect on the polarity or charge of amino acid residues at this position. Conservative amino acid substitutions also include non-naturally occurring amino acid residues that are commonly incorporated by chemical peptide synthesis rather than by a biological system synthesis. These include peptidomimetics and other reverse forms of amino acid portion.

The naturally occurring residues can be grouped into the following classes based on common side chain properties:
(1) Hydrophobic: norleucine, M, A, V, L, I;
(2) Neutral hydrophilicity: C, S, T;
(3) Acid: D, E;
(4) Alkaline: N, Q, H, K, R;
(5) Residues affecting chain orientation: G, P; and
(6) Aromatic: W, Y, F.

Conservative substitutions may include one member of these categories being exchanged for another member of the same category.

Some conservative amino acid substitutions are shown in the following table:

| Amino | |
|---|---|
| acid | conservative amino acid substitutions |
| Ala | D-Ala, Gly, Aib, β-Ala, L-Cys, D-Cys |
| Arg | D-Arg, Lys, Orn, D-Orn |
| Asn | D-Asn, Asp, D-Asp, Glu, D-Glu, Gln, D-Gln |
| Asp | D-Asp, D-Asn, Asn, Glu, D-Glu, Gln, D-Gln |
| Cys | D-Cys, S-Me-Cys, Met, D-Met, Thr, D-Thr, L-Ser, D-Ser |
| Gln | D-Gln, Asn, D-Asn, Glu, D-Glu, Asp, D-Asp |
| Glu | D-Glu, Asp, D-Asp, Asn, D-Asn, Gln, D-Gln |
| Gly | Ala, D-Ala, Pro, D-Pro, Aib, β-Ala |
| Ile | D-Ile, Val, D-Val, Leu, D-Leu, Met, D-Met |
| Leu | D-Leu, Val, D-Val, Met, D-Met, Ile, D-Ile |
| Lys | D-Lys, Arg, D-Arg, Orn, D-Orn |
| Met | D-Met, S-Me-Cys, Ile, D-Ile, Leu, D-Leu, Val, D-Val |
| Phe | D-Phe, Tyr, D-Tyr, His, D-His, Trp, D-Trp |
| Pro | D-Pro |
| Ser | D-Ser, Thr, D-Thr, allo-Thr, L-Cys, D-Cys |
| Thr | D-Thr, Ser, D-Ser, allo-Thr, Met, D-Met, Val, D-Val |
| Tyr | D-Tyr, Phe, D-Phe, His, D-His, Tip, D-Trp |
| Val | D-Val, Leu, D-Leu, Ile, D-Ile, Met, D-Met |

Alternatively, the following table lists examples of conservative amino acid substitutions, 0 or more represents that two amino acids are conservative amino acid substitutions between each other:

| | C | G | P | S | A | T | D | E | N | Q | H | K | R | V | M | I | L | F | Y | W |
|---|---|---|---|---|---|---|---|---|---|---|---|---|---|---|---|---|---|---|---|---|
| W | −8 | −7 | −6 | −2 | −6 | −5 | −7 | −7 | −4 | −5 | −3 | −3 | 2 | −6 | −4 | −5 | −2 | 0 | 0 | 17 |
| Y | 0 | −5 | −5 | −3 | −3 | −3 | −4 | −4 | −2 | −4 | 0 | −4 | −5 | −2 | −2 | −1 | −1 | 7 | 10 | |
| F | −4 | −5 | −5 | −3 | −4 | −3 | −6 | −5 | −4 | −5 | −2 | −5 | −4 | −1 | 0 | 1 | 2 | 9 | | |
| L | −6 | −4 | −3 | −3 | −2 | −2 | −4 | −3 | −3 | −2 | −2 | −3 | −3 | 2 | 4 | 2 | 6 | | | |
| I | −2 | −3 | −2 | −1 | −1 | 0 | −2 | −2 | −2 | −2 | −2 | −2 | −2 | 4 | 2 | 5 | | | | |
| M | −5 | −3 | −2 | −2 | −1 | −1 | −3 | −2 | 0 | −1 | −2 | 0 | 0 | 2 | 6 | | | | | |
| V | −2 | −1 | −1 | −1 | 0 | 0 | −2 | −2 | −2 | −2 | −2 | −2 | −2 | 4 | | | | | | |
| R | −4 | −3 | 0 | 0 | −2 | −1 | −1 | −1 | 0 | 1 | 2 | 3 | 6 | | | | | | | |
| K | −5 | −2 | −1 | 0 | −1 | 0 | 0 | 0 | 1 | 1 | 0 | 5 | | | | | | | | |
| H | −3 | −2 | 0 | −1 | −1 | −1 | 1 | 1 | 2 | 3 | 6 | | | | | | | | | |
| Q | −5 | −1 | 0 | −1 | 0 | −1 | 2 | 2 | 1 | 4 | | | | | | | | | | |
| N | −4 | 0 | −1 | 1 | 0 | 0 | 2 | 1 | 2 | | | | | | | | | | | |
| E | −5 | 0 | −1 | 0 | 0 | 0 | 3 | 4 | | | | | | | | | | | | |
| D | −5 | 1 | −1 | 0 | 0 | 0 | 4 | | | | | | | | | | | | | |
| T | −2 | 0 | 0 | 1 | 1 | 3 | | | | | | | | | | | | | | |
| A | −2 | 1 | 1 | 1 | 2 | | | | | | | | | | | | | | | |
| S | 0 | 1 | 1 | 1 | | | | | | | | | | | | | | | | |
| P | −3 | −1 | 6 | | | | | | | | | | | | | | | | | |
| G | −3 | 5 | | | | | | | | | | | | | | | | | | |
| C | 12 | | | | | | | | | | | | | | | | | | | |

When forming the fusion protein of the invention, it may, but not necessarily, use a linker or a connector. When a linker is present, the chemical structure of it may be flexible since it primarily functions as a spacer. Linkers may be composed of amino acids linked together by peptide bonds. In some embodiments of the invention, the linker consists of 1 to 20 amino acids linked by peptide bonds. In some embodiments, the 1 to 20 amino acids are selected from the 20 naturally occurring amino acids. In various embodiments, the 1 to 20 amino acids are selected from consists of multiple amino acids that are spatially unhindered. In other embodiments, the spatially unhindered amino acids consist of glycine and alanine. The G-rich polypeptides of the present invention may be selected from (G) 3-S, i.e., “GGGS”, (G) 4-S, i.e., “GGGGS” and (G) 5-S, i.e., “GGGGGS”. In some embodiments, the linker comprises GGGGSGGGGS (SEQ ID NO: 32), GGGGSGGGGSGGGGS (SEQ ID NO: 33) or GGGGSGGGGGGGGSA (SEQ ID NO: 33). Other suitable linkers include: GGGGGSGGGSGGGGS (SEQ ID NO: 34), GGGKGGGG (SEQ ID NO: 35), GGGNGSGG (SEQ ID NO: 36), GGGCGGGG (SEQ ID NO: 37) and GPNGG (SEQ ID NO: 38). Although a 15 amino acid residue linker was found to play a particularly good role for the dual-target fusion protein, the present invention contemplates linkers of any length or composition.

Linkers described herein are exemplary, and the linker disclosed herein may be much longer and may contain other residues. Linkers in the present invention may also be non-peptide linkers. For example, an alkyl linker can be used, such as —NH—(CH₂)ₛ—C(O)—, wherein s=2-20. These alkyl linkers may be further substituted with any non-sterically hindered group including, but not limited to, lower alkyl (e.g., C1-C6), lower acyl, halogen (e.g., Cl, Br), CN, NH₂ or phenyl. An exemplary non-peptidic linker can also be a polyethylene glycol linker, wherein the linker has a molecular weight of 100-5000 kD, such as 100-500 kD.

As used herein, “one or more” amino acid substitutions is defined where “more” means greater than one, such as from 1 to 30, 1 to 20, 1 to 10, 1 to 5, or such as 1, 2, 3, 4, 5, 6, 7, 8, 9, 10, 11, 12, 13, 14, 15, 16, 17, 18, 19, 20, 21, 22, 23, 24, 25, 26, 27, 28, 29 and 30.

The “first” and “second” described in this article are only for the purpose of distinguishing the description and have no special meaning.

The “ADCC effect” described herein is antibody-dependent cellular cytotoxicity (ADCC); human IgG subclasses (G1, G2, G3, G4) have different biological activities (referred to as effector functions). These effector functions are often mediated by interaction with the FcγR receptor (FcγR) or by binding to complement 1 (C1q) sub-component, wherein the complement 1 sub-component recognizes and binds to the heavy chain of immunoglobulin G or immunoglobulin M, initiating the classic complement pathway. Binding to FcyR can result in antibody-dependent cell-mediated cytolysis, whereas binding to complement factors can result in complement-mediated cytolysis, i.e., complement-dependent cytotoxicity (CDC).

As used herein, the term “comprising” usually means the recited elements but not excluding other elements.

The terms “protein” and “polypeptide” are used interchangeably and, in their broadest sense, refer to a compound of two or more subunits amino acids, amino acid analogs or peptidomimetics. Subunits can be linked by peptide bonds. In another embodiment, the subunits may be linked by other bonds, such as esters, ethers, amino groups, and the like. The protein or polypeptide must contain at least two amino acids and there is no limit to the maximum number of amino acids that can make up the protein or peptide sequence. The term “amino acid” as used herein refers to natural and/or unnatural or synthetic amino acids including D and L optical isomers of amino acids, such as glycine, D and L optical isomers, amino acid analogs and peptide mimetics.

“Homology” or “identity” or “similarity” refers to the sequence similarity between two peptides or between two nucleic acid molecules. Homology can be determined by comparing the positions in each sequence that can be aligned for comparative purposes. When the positions in the compared sequences are occupied by the same base or amino acid, these molecules are homologous at that position. The degree of homology between sequences varies with the number of matches or homologous positions shared by the sequences. An “unrelated” or “non-homologous” sequence shares less than 40% identity, or alternatively less than 25% identity with one of the sequences of the disclosure.

The term “at least 80% sequence identity” with respect to polypeptide sequence comparisons is used throughout this specification. The expression usually refers to at least 80%, at least 81%, at least 82%, at least 83%, at least 84%, at least 85%, at least 86%, at least 87%, at least 88%, at least 89%, at least 90% %, at least 91%, at least 92%, at least 93%, at least 94%, at least 95%, at least 96%, at least 97%, at least 98% or at least 99% sequence identity to each reference sequence.

When used in connection with a numerical value, the term “about” is intended to include numerical values in the range having a lower limit of 5% less than the indicated value and an upper limit of 5% greater than the indicated value.

The term “fusion protein” generally refers to a protein resulting from two or more proteins or polypeptides. A gene or nucleic acid molecule encoding two or more proteins or polypeptides can be joined to form a fusion gene or fusion nucleic acid molecule. The fusion gene or fusion nucleic acid molecule can encode the fusion protein. Translation of the fusion gene produces a single polypeptide having the property of at least one, or even each, of the two or more proteins or polypeptides prior to fusion. Recombinant fusion proteins are artificially created by recombinant DNA techniques for biological research or therapy. Recombinant fusion proteins are created by genetically engineering fusion genes. The present invention relates to recombinant fusion proteins, and the terms fusion proteins and recombinant fusion proteins are used herein with the same meaning. The fusion proteins described herein generally comprise at least two domains (A and C), and optionally a third component, a linker between the two domains. Generation of recombinant fusion proteins is known in the art and generally involves removing the stop codon from the self-encoding the cDNA sequence of the first protein or polypeptide. The cDNA sequence for the second protein is then attached in-frame by ligation or overlap extension PCR. Finally, the DNA sequence is expressed by the cell as a single protein. The protein can be engineered to include the complete sequence of two original proteins or polypeptides, or just a fraction of either.

As used in this specification and claims, the singular forms “a”, “an” and “the” include plural referents unless the context clearly dictates otherwise. For example, the term “pharmaceutically acceptable carrier” includes a plurality of pharmaceutically acceptable carriers, including mixtures thereof.

“Composition” usually is a combination of two or more substances, for example, a combination of the active agent with other inert or active compounds.

“Pharmaceutical composition” usually includes the active agent in combination with an inert or active carrier, so that the composition is suitable for diagnostic or therapeutic uses, either in vivo or in vitro or ex vivo.

In the present invention, the term “therapeutically effective amount” generally refers to the minimum dose of active ingredient required to produce a therapeutic benefit in a subject. For example, for a patient exhibiting or susceptible to type 2 diabetes, obesity, or metabolic syndrome, or for preventing the onset of the disease, “therapeutically effective amount” refers to a dose that is capable of inducing, ameliorating, or causing a pathological condition, disease progression, or physiological condition that is associated with or counteracted by the disorder described above. In the present application, the term “subject” or “patient” may be human, but may also be a non-human animal, more specifically may be a companion animal such as a dog, a cat or the like, a farm animal such as a cow, a sheep, a pig, horses, or laboratory animals such as rats, mice, guinea pigs, and the like.

In the present invention, an expression of “XnY” indicates that residue X at position n in a sequence is substituted with residue Y when describing amino acid residues substitution in the sequence. For example, amino acid substitution of “R175L” indicates that residue R at position 175 in a sequence is substituted with residue L.

### FUSION PROTEIN, PREPARATION METHOD AND APPLICATION

The inventors of the present invention found that known two-target fusion proteins can not completely avoid the problem of dimer dissociation. The reason may be that interactions occurring in the 3D structure of 200-amino-acid chains increase to the other end of the Fc of the double-target fusion protein; thus, more stable IgG4 Fc portion structure is required to prevent dimer dissociation.

On the basis of a large amount of inventive work by the inventors, it was surprisingly found that when two mutations (S228P and R409K) were introduced into the Fc of IgG4, the stability of the IgG4 Fc portion structure was greatly enhanced. Also, the mutated Fc had lower (or absolutely no) ADCC or CDC effects than the commonly used Fc. Therefore, the present invention provides a fusion protein structure with high stability and low ADCC/CDC effects.

In one aspect of the invention, the invention provides a fusion protein comprising or consisting of:

A-L1-B-L2-C or C-L1-B-L2-A,

Wherein A is a GLP-1 receptor agonist comprising the amino acid sequence set forth in SEQ ID NO: 1 (HGEGTFTSDV SSYLEEQAAK EFIAWLVKGG G) or at least about 80% identity to the amino acid sequence set forth in SEQ ID NO: 1, or at least about 90% identity to the amino acid sequence shown in SEQ ID NO: 1, or at least about 95% identity to the amino acid sequence set forth in SEQ ID NO: 1, or at least about 98% identity to the amino acid sequence set forth in SEQ ID NO: 1, or at least about 99% identity to the amino acid sequence set forth in SEQ ID NO: 1;

B is an IgG4-Fc mutant, comprising the amino acid sequence of SEQ ID NO: 2 (ESKYGPPCPP CPAPEAAGGP SVFLFPPKPK DTLMISRTPE VTCVVVDVSQ EDPEVQFNWY VDGVEVHNAK TKPREEQFNS TYRVVSVLTV LHQDWLNGKE YKCKVSNKGL PSSIEKTISK AKGQPREPQV YTLPPSQEEM TKNQVSLTCL VKGFYPSDIA VEWESNGQPE NNYKTTPPVL DSDGSFFLYS KLTVDKSRWQ EGNVFSCSVM HEALHNHYTQ KSLSLSLG) or at least about 95% identity to the amino acid sequence of SEQ ID NO: 2, or at least about 98% identity to the amino acid sequence set forth in SEQ ID NO: 2, or at least about 99% identity to the amino acid sequence shown in SEQ ID NO: 2, wherein, B comprises the tenth amino acid residue of P (EU number corresponding to 228) and the 191^{th} amino acid residue of K (EU number corresponding 409);

C is FGF21, comprising the amino acid sequence set forth in SEQ ID NO: 6 (HPIPDSSPLL QFGGQVRQRY LYTDDAQQTE AHLEIREDGT VGGAADQSPE SLLQLKALKP GVIQILGVKT SRFLCQRPDG ALYGSLHFDP EACSFRERLL EDGYNVYQSE AHGLPLHLPG NKSPHRDPAP RGPARFLPLP GLPPALPEPP GILAPQPPDV GSSDPLSMVG GSQGRSPSYA S) or at least about 90% identity to the amino acid sequence set forth in SEQ ID NO: 6, or at least about 95% identity to the amino acid sequence set forth in SEQ ID NO: 6, or at least about 98% identity to the amino acid sequence set forth in SEQ ID NO: 6, or a polypeptide having at least about 99% identity to the amino acid sequence set forth in SEQ ID NO: 6 and having FGF21 activity; and

L1 and/or L2 are a polypeptide comprising 5 to 50 amino acid residues and the polypeptide comprises at least 50% G.

In some embodiments, A comprises or consists of the amino acid sequence of SEQ ID NO: 7 (HX₁EGTFTSDVSSYLEX₂QAAK EFIAWLX₃X₄GX₅X₆) (SEQ ID NO: 7),

Wherein, X₁ is selected from G or V;
X₂ is selected from E or G;
X₃ is selected from V or K;
X₄ is selected from K or N;
X₅ is selected from G or E; and
X₆ is selected from G or P or absent.

In other embodiments, A comprises or consists of the amino acid sequence set forth in SEQ ID NO: 1.

In some embodiments, B comprises or consists of the amino acid sequence set forth in SEQ ID NO: 8,
ESKYGPPCPP CPAPX₇X₈X₉GGP SVFLFPPKPK DTLMISRTPE VTCVVVDVSQ EDPEVQFNWY VDGVEVHNAK TKPREEQFX₁₀S TYRVVSVLTV LHQDWLNGKE YKCKVSNKGL PSSIEKTISK AKGQPREPQV YTLPPSQEEM TKNQVSLTCL VKGFYPSDIA VEWESNGQPE NNYKTTPPVL DSDGSFFLYS KLTVDKSRWQ EGNVFSCSVM HEALHNHYTQ KSLSLSLGX₁₁ (SEQ ID NO: 8)

Wherein, X₇ is selected from P or E;
X₈ is selected from F, V or A;
X₉ is selected from L, E or A;
X₁₀ is selected from N or A; and
X₁₁ is selected from K or absent.

In other embodiments, B comprises or consists of the amino acid sequence set forth in SEQ ID NO: 2.

In some embodiments, C comprises or consists of the amino acid sequence set forth in SEQ ID NO: 9,
HPIPDSSPLL QFGGQVRQRY LYTDDAQQTE AHLEIREDGT VGGAX₁₂DQSPE SLLQLKALKP GVIQILGVKT SRFLCQRPDG ALYGSLHFDP EACSFREX₁₃LX₁₄ EDGYNVYQSE AHGLPLHX₁₅PG NKSPHRDPAP RGPX₁₆RFLPLP GLPPALPEPX₁₇ X₁₈X₁₉LAPQPPDV GSSDPLX₂₀MVX₂₁ X₂₂X₂₃QG X₂₄SPSX₂₅X₂₆ S (SEQ ID NO: 9)

Wherein, X₁₂ is selected from A, K, R, E or Q;
X₁₃ is selected from L, D, R, E, Q, K or T;
X₁₄ is selected from L or K;
X₁₅ is selected from L or C;
X₁₆ is selected from A or C;
X₁₇ is selected from P or A;
X₁₈ is selected from G or A;
X₁₉ is selected from I or V;
X₂₀ is selected from S, H, C or R;
X₂₁ is selected from G, E, A, D or S;
X₂₂ is selected from P, A, E, Q, W, C, G or T;
X₂₃ is selected from S, L, V or M;
X₂₄ is selected from R, L, H or P;
X₂₅ is selected from Y, S, A or F; and
X₂₆ is selected from A, S, E or G.

In other embodiments, the C further comprises:
a) amino-terminal truncation of not more than 8 amino acid residues;
b) carboxy-terminal truncation of not more than 12 amino acid residues;
c) amino-terminal truncation of not more than 8 amino acid residues and carboxy-terminal truncation of not more than 12 amino acid residues; or
d) carboxy-terminal extension of no more than 6 amino acid residues.

In some embodiments, L1 and/or L2 comprise 1, 2 or 3 G-rich polypeptides, in some embodiments, L1 and/or L2 are selected from GGGGSGGGGS (SEQ ID NO: 32) or GGGGSGGGGSGGGGS (SEQ ID NO: 13) or GGGGSGGGGSGGGGSA (SEQ ID NO: 33).

In some embodiments, the fusion protein is homodimers.

In other embodiments, the fusion protein is non-homodimers.

The natural FGF21 molecule contains a site susceptible to protease hydrolysis; its half-life in vivo is very short. Since the three-dimensional structure of FGF21 has not been solved, researchers have made various attempts to reform its structure in anticipation of obtaining a longer half-life. These attempts include the truncation, elongation, substitution, insertion or deletion of certain amino acids on the basis of a natural amino acid sequence. Some researchers even mutate sites one by one and look forward to finding the key core that affects activity and half-life.

The present invention provides a dual-target fusion protein of GLP-1 and FGF21, which has a mutation site different from the existing published fusion protein. The inventor has unexpectedly found that the fusion protein provided herein has a longer half-life and can be administered for more than one week. Even more gratifying is that the fusion protein can exert a synergistic effect, has a remarkably stable hypoglycemic effect, and has good biological activities of lowering lipid and reducing body weight.

In another aspect of the invention, the invention provides a homodimeric fusion protein comprising or consisting of:

A-L1-B-L2-C,

Wherein, A is a GLP-1 receptor agonist comprising the amino acid sequence set forth in SEQ ID NO: 10 (HX₁EGTFTSDV SSYLEX₂QAAK EFIAWLVKGX₅G), wherein X₁ is selected from G or V; X₂ is selected from E or G; and X₅ is selected from G or E.

B is an IgG4-Fc variants comprising SEQ ID NO: 11 (ESKYGPPCPP CPAPEX₈X₉GGP SVFLFPPKPK DTLMISRTPE VTCVVVDVSQ EDPEVQFNWY VDGVEVHNAK TKPREEQFNS TYRVVSVLTV LHQDWLNGKE YKCKVSNKGL PSSIEKTISK AKGQPREPQV YTLPPSQEEM TKNQVSLTCL VKGFYPSDIA VEWESNGQPE NNYKTTPPVL DSDGSFFLYS KLTVDKSRWQ EGNVFSCSVM HEALHNHYTQ KSLSLSLGX₁₁), wherein X₈ is selected from F, V or A; X₉ is selected from L, E or A; X₁₁ is selected from K or absent.

C is FGF21, comprising SEQ ID NO: 12 (HPIPDSSPLL QFGGQVRQRY LYTDDAQQTE AHLEIREDGT VGGAVDQSPE SLLQLKALKP GVIQILGVKT SRFLCQRPDG ALYGSLHFDP EACSFREX₁₃LL EDGYNVYQSE AHGLPLHLPG NKSPHRDPAP RGPARFLPLP GLPPALPEPP GILAPQPPDV GSSDPL X₂₀MVG X₂₂SQG X₂₄SPSX₂₅A S)

Wherein X₁₃ is selected from L, D, R, E;
X₂₀ is selected from S, H or C;
X₂₂ is selected from A or G;
X₂₄ is selected from R, L or P; and

The amino acid mutation at position X₂₅ is selected from any one of the following: Y, S, F.

L1 and/or L2 are a polypeptide comprising 5 to 50 amino acid residues and the polypeptide comprises at least 50% G.

In some embodiments, A comprises or consists of the amino acid sequence set forth in SEQ ID NO: 1.

In some embodiments, B comprises or consists of the amino acid sequence set forth in SEQ ID NO: 2.

In some embodiments, the homodimeric fusion protein comprises or consists of the amino acid sequence set forth in SEQ ID NO: 5, 17, 18, 19, 20, 21.

In another aspect of the invention, the invention provides polynucleotides encoding the fusion proteins of the invention. The polynucleotide encoding the above protein may be in the form of RNA, or in the form of DNA, including cDNA and synthetic DNA. The DNA may be double-stranded or single-stranded. The sequence encoding the protein of the invention may be different due to the redundancy or degeneracy of the genetic code. Polynucleotides encoding proteins of the invention may comprise the following: only proteins coding sequences, proteins coding sequences and other coding sequences such as leader or secretory sequences or preprotein sequences; proteins coding sequences and non-coding sequences such as intron or 5′ and/or 3′ non-coding sequences of an protein coding sequence. Thus, the term “polynucleotide encoding a protein” encompasses polynucleotides which may include not only proteins coding sequences but also other coding sequences and/or non-coding sequences.

In a further aspect of the invention, there is also provided a vector comprising a polynucleotide according to the invention. The expression vector usually replicates in the host organism either as an additional body or as an integral part of the host's chromosomal DNA. In general, expression vectors contain selection markers such as tetracycline, neomycin, and dihydrofolate reductase to allow detection of those cells transformed with the desired DNA sequence.

In a further aspect of the invention, there is also provided a host cell comprising a vector according to the invention. The dual-target protein of the present invention can easily be produced in mammalian cells such as CHO, NSO, HEK293 or COS cells, in bacterial cells such as E. coli, Bacillus subtilis, Pseudomonas fluorescence, or in fungal or yeast cells. Host cells (for example HEK293) are cultured using techniques known in the art.

In a further aspect of the invention, there is also provided a method of preparing a fusion protein according to the invention; the method comprises expressing a vector according to the invention in a host cell. The vector containing the polynucleotide sequence of interest (eg, the dual-target fusion protein and the expression control sequence) can be transferred to the host cell by well-known methods, and such methods vary depending on the type of host cell. For example, calcium chloride transformation is commonly used for prokaryotic cells, whereas calcium phosphate treatment or electroporation can be used for other host cells.

In another aspect of the present invention, there is also provided use of the fusion protein of the present invention in the manufacture of a pharmaceutical composition, the pharmaceutical composition comprises a fusion protein of the invention, and at least one pharmaceutically acceptable carrier, diluent or excipient. In some embodiments, the medicament is used for the treatment of metabolic diseases. In some embodiments, the metabolic disease is diabetes, such as type 2 diabetes. In other embodiments, the metabolic disease is obesity. Other embodiments include metabolic disorders such as dyslipidemia, hypertension, hepatic steatosis (for example non-alcoholic fatty liver (NASH)), cardiovascular diseases (for example atherosclerosis and aging).

### EXAMPLE

The disclosure is further understood by the following examples, which are merely examples of the disclosure. The present disclosure is not to be limited in scope by the illustrated embodiments, which are merely intended to illustrate a single aspect of the present invention. Any method that is functionally equivalent is included in the scope of the present invention. From the above description and the drawings, various modifications of the invention in addition to those described herein will be apparent to those skilled in the art. These modifications are also within the scope of the claims.

### Example 1: Construction of Vector

The fusion protein carrier was constructed by using a molecular cloning method, the mutants and sequences of the fusion proteins were shown in Table 1.

TABLE 1

| Fusion | | | | SEQ ID |
|---|---|---|---|---|
| protein | GLP-1 portion | Fc portion | FGF21 portion | NO: |
| Dulaglutide | A8G\G22E\R36G | S228P\F234A\L235A | non | 3 |
| S1F1 | non | S228P\F234A\L235A\R409K | L98R\P171G | 4 |
| D1F1 | A8G\G22E\R36G | S228P\F234A\L235A\R409K | L98R\P171G | 5 |
| D1F2 | A8G\G22E\R36G | S228P\F234A\L235A | L98R\P171G | 16 |
| D2F1 | A8G\G22E\R36G | 8228P\F234A\L235A\R409K | L98R\P171G\Y179F | 17 |
| D3F1 | A8G\G22E\R36G | 8228P\F234A\L235A\R409K | L98R\S167H\P171A | 18 |
| D4F1 | A8G\G22E\R36G | 8228P\F234A\L235A\R409K | L98R\S167H\P171A\R175L | 19 |
| D5F1 | A8G\G22E\R36G | 8228P\F234A\L235A\R409K | L98R\S167H\P171A\R175L | 20 |
| D6F1 | A8G\G22E\R36G | 8228P\F234A\L235A\R409K | L98R\G170E\P171A\S172L | 21 |
| D7F1 | A8G\G22E\R36G | 8228P\F234A\L235A\R409K | wild type | 22 |

The nucleotide sequence encoding dulaglutide and the S1F1 fusion protein was synthesized by Kingsbury Biotechnology Co., Ltd. through chemical methods. The nucleotide sequence encoding D1F1 fusion protein was obtained by connecting fragments of nucleotide sequences respectively encoding GLP-1 and Fc-FGF21 by SOE-PCR technique, the nucleotide sequences respectively encoding GLP-1 and Fc-FGF21 were obtained by PCR through designing a primer and using dulaglutide and S1F1 fusion protein nucleotide sequences as templates.

The nucleotide sequences encoding D1F2, D2F1, D3F1, D4F1, D5F1, D6F1 and D7F1 fusion proteins were synthesized by Suzhou Genewiz Biological Technology Co., Ltd through chemical methods.

The nucleotide sequence and the vector plasmid pcDNA3.4 were digested with endonucleases HindIII and EcoRI (TAKARA, Japan) at 37° C. The digested products were recovered by purification using the Gel Extraction Kit (OMEGA, America) according to the manufacturer's instructions. The purified target gene was ligated with the vector using a DNA Ligation Kit Ver.2.1 (TAKARA, Japan) according to the manufacturer's instructions and incubated at 16° C. for 1 hour to obtain a recombinant expression plasmid.

The above recombinant plasmid was transformed into competent DH5a cells. Bacteria was coated into an ampicillin plate. The monoclonal on the plate was picked and cultured in 1 ml of LB medium (peptone 10 g/L, yeast extract 5 g/L, sodium chloride 10 g/L and agar 2%, antibiotic content 100 g/mL) to extract the plasmid. After sequencing and validation, a series of validated correct expression vectors were extracted with Invitrogen Plasmid Kit and digested with restriction enzyme PvuI (TAKARA, Japan). After linearization, the product was purified by ethanol precipitation method and stored at −20° C. for future use.

### Example 2: Transfection of Encoding Dulaglutide Vector and Expression in Cells

After CHOK1SV GS-KO (Lonza) host cells were resuscitated with CD CHO medium (gibco), cells were collected and transfected at a cell density of about 8×10⁵ cells/mL. The cells were transfected with about 1×10⁷ cells and the vector was about 40 μg and transfected by means of electric shock (Bio-Rad, Gene PulS Xcell). After shock, the cells were cultured in 20 mL of CD CHO medium. On the second day of culture, the cells were collected by centrifugation at 200 g for 10 min. The cells were resuspended and cultured in 20 mL of CD CHO medium with a final concentration of 50 μM obtained by addition of MSX (sigma). When the cell density was about 0.6×10⁶ cells/mL, the obtained mixed clone was passaged with CD CHO medium and the passage cell density was about 0.2×10⁶ cells/mL. When the cell viability was about 90%, the cell culture fluid was collected.

### Example 3: Transfection of S1F1 and D1F1, D1F2, D2F1, D3F1, D4F1, D5F1, D6F1, D7F1 Fusion Protein Vectors and Expression in Cells

After HEK293F host cells (Invitrogen, Freestyle 293F) were resuscitated with 293 Expression Medium (Invitrogen), they were collected and transfected at a cell density of about 1×10⁶ cells/mL. The cells were transfected with FreeStyle™ MAX Reagent Transfection Kit. There were about 3×10⁷ cells and the vector was about 37.5 μg. After transfection, the cells were cultured in 30 mL of 293 Expression Medium. On the second day of culture, genetic transformants started to screen with geneticin (merck). As needed due to cell growth, medium was replaced every 3 to 5 days for screening. After about 14 days of selection, resistant clones appeared and could be expanded. The cell passage density was about 0.5×10⁶ cells/mL. The obtained mixed clone was subcultured with 293 Expression Medium. When the cell viability was about 90%, the cell culture fluid was collected.

### Example 4: Purification of Protein From the Collected Cell Fermentation Broth

The translation level of the eight fusion proteins described in Examples 2 and 3 were detected. The collected cell culture medium was purified by Protein A chromatography (EzFast Protein A Diamond, Bestchrom). The equilibration solution was 20 mM PBS, 0.15 M NaCl, pH 7.4. The fusion protein dulaglutide and the S1F1 were eluted with 0.1 M citric acid buffer, pH 3.2 +0.2. D1F1, D1F2, D2F1, D3F1, D4F1, D5F1, D6F1 and D7F1 fusion proteins were eluted with 0.1 M glycine pH 3.2±0.2. The target eluate was collected at target absorption peak and dialyzed with PBS buffer to take part of the sample for mass spectrometry. Mass spectrometry detection molecular weight was consistent with theoretical molecular weight, and they were in homodimeric form. The fusion protein was confirmed to be the target fusion protein. At the same time, the collected samples were detected by 10% SDS-PAGE electrophoresis after reduction and non-reduction. Electrophoresis showed a single band having a band size consistent with theory.

### Example 5: Detection of cAMP Produced by Fusion Protein Stimulation in HEK293/GLP-1R/KLB Cell Model

cAMP was produced by HEK293 cells with GLP-1R/KLBGLP-1 expression stimulated by the test fusion protein. The cAMP assay kit (Cisbio 62AM6PEC) was used to measure the concentration of the fusion protein and the amount of cAMP produced by stimulation to establish a dose-response curve measuring the activity of the test compounds in vitro. Two fusion proteins were tested, including the dulaglutide and D3F1 fusion protein groups. cAMP test results are shown in FIG. 2 and Table 2.

TABLE 2

| The test protein | Dulaglutide | D3F1 |
|---|---|---|
| EC50 | 0.2683 | 0.01984 |
| Amino acid sequence | SEQ ID NO: 3 | SEQ ID NO: 18 |

### Example 6: Investigation of the Effect of Mutation of Fc409 From R to K on the Stability of the Fusion Protein by Microcalorimetric Differential Scanning calorimetry (DSC)

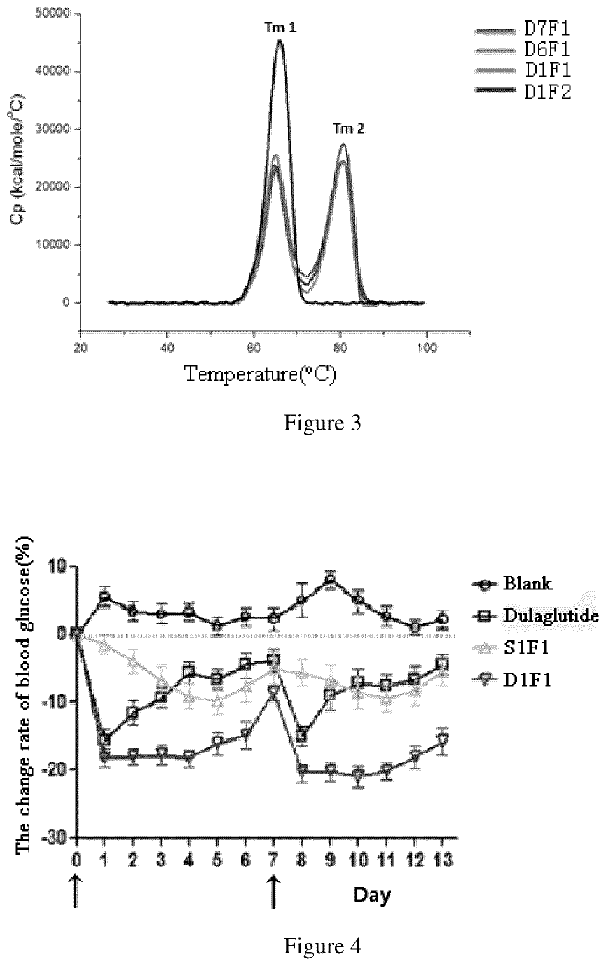

The fusion proteins D1F1, D1F2, D6F1 and D7F1 obtained in Example 4 were each diluted to 1 mg/ml with a blank buffer system (20 mM PBS pH 7.4), taking 500 μL for DSC (Microcal vp-Capillary DSC) analysis. The scanning temperature was from 20° C. to 100° C. and the heating rate was 1° C./min. The microcalorimetric scanning calorimetry results are shown in FIG. 3, and the obtained Tm values after fitting with Origin™ are shown in Table 3.

TABLE 3

| | Protein | | | |
|---|---|---|---|---|
| Test | concentration | DH | Tm1 | Tm2 |
| protein | (nM) | (enthalpy) | (° C.) | (° C.) |
| D7F1 | About 9.7 | 3.56E+08 | 65.43 | 80.74 |
| D6F1 | | 3.28E+08 | 64.73 | 80.7 |
| D1F1 | | 3.12E+08 | 64.93 | 80.58 |
| D1F2 | | 2.85E+08 | 65.71 | N/A |

The higher the phase transition temperature Tm, the stronger the molecular stability. It can be seen from the above results that the Tm1 and Tm2 values of the samples D7F1, D6F1 and D1F1 are basically the same, indicating that the stability of the three proteins is more consistent. The Tm1 value of D1F2 is basically the same as that of the other three samples, but its Tm2 is significantly lower Tm2 values for other samples. From the structural analysis of the D1F1 and D1F2 proteins, it can be concluded that the main difference is that the R at amino acid 409 in CH3 domain of IgG4 Fc is mutated to K, and this mutation resulted in a significantly higher Tm2 for D1F1 than the Tm2 value for D1F2. The results showed that the mutation from R to K at position 409 in the CH3 domain of IgG4 Fc in D1F1 increased the Tm value of the domain and thus enhanced its protein stability.

### Example 7: A Study of the Effect of Fc409 Mutation From R to K on the Hypoglycemic Effect of the Fusion Protein

The efficacy of the fusion protein in the db/db mouse model (Institute of Model Animal Science, Nanjing University) was studied. The mice were randomly divided into three groups, including blank group, D1F1 group and D1F2 group. Each db/db mouse was injected with 24.5 nM/kg fusion protein, the administration volume was 10 ml/kg. Seven mice were used per fusion protein and administrated by once injection, and the blood glucose level was measured at different times. The results are shown in Table 4. As a result, the 409^{th} of IgG4 Fc was mutated to K, hypoglycemic effect could maintain longer.

TABLE 4

| | blood glucose concentration (mmol/L) | | | |
|---|---|---|---|---|
| Group | Day 0 | Day 2 | Day5 | Day 8 |
| Blank group | 24.0 ± 5.1 | 21.0 ± 6.5 | 23.8 ± 6.1 | 19.8 ± 5.4 |
| D1F1 | 24.0 ± 5.2 | 8.7 ± 2.5 | 15.2 ± 4.5 | 16.5 ± 5.2 |
| D1F2 | 24.5 ± 4.3 | 8.4 ± 2.5 | 17.3 ± 5.5 | 19.5 ± 3.7 |

### Example 8: A Study of the Hypoglycemic Effect of Dulaglutide, S1F1, D1F1 in db/db Mouse Model

Efficacy of fusion proteins in db/db mouse model (Institute of Model Animal Science, Nanjing University) was studied. A series of purified fusion proteins were diluted with 10 mM PBS during the experiment. The mice were randomly divided into four groups, including blank group, dulaglutide group, S1F1 group and D1F1 group. Each db/db mouse was injected with 40 nM/kg fusion protein, the administration volume was 3 ml/kg, and 10 mice per fusion protein were injected once a week for two weeks. The results of sampling changes in blood glucose after administration are shown in FIG. 4. The D1F1 group had a significant hypoglycemic advantage and its hypoglycemic curve was more stable than both the dulaglutide and S1F1 groups.

### Example 9: A Study of the Hypoglycemic Effect of D1F1, D2F1, D7F1 in db/db Mouse Model

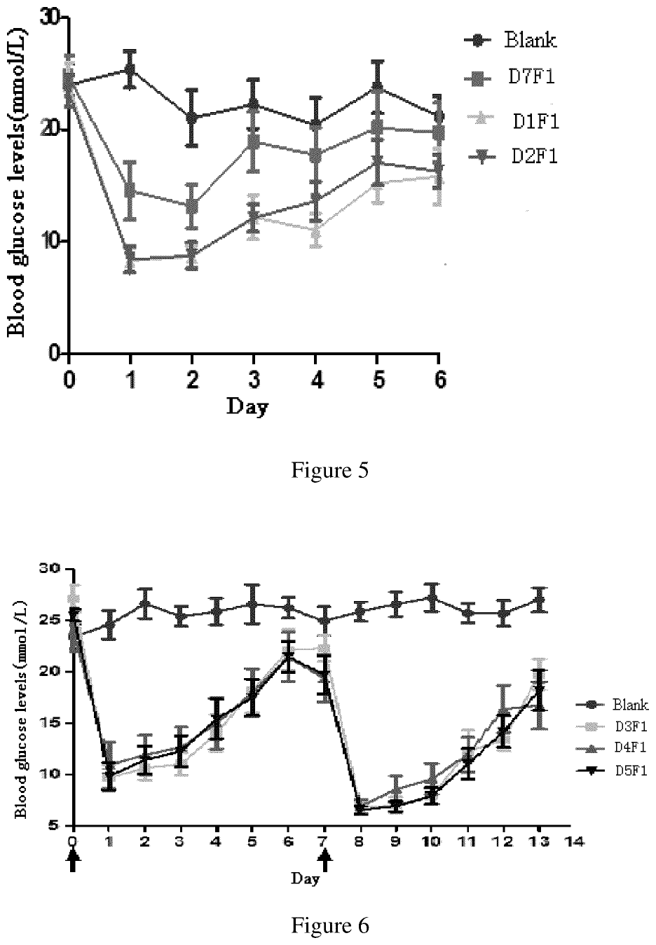

The efficacy of the fusion protein in the db/db mouse model (Institute of Model Animal Science, Nanjing University) was studied. The mice were randomly divided into four groups, including blank group (10 mM PBS), D1F1 group, D2F1 group and D7F1 group. Each db/db mouse was injected with 24.5 nM/kg fusion protein (Diluted with 10 mM PBS), the administration volume was 10 ml/kg. Seven mice per fusion protein were injected once, and the blood glucose level was measured at different times. The results are displayed in FIG. 5. They showed that D1F1 and D2F1 had better hypoglycemic effects than D7F1.

### Example 10: A Study of the Hypoglycemic Effect of D3F1, D4F1, D5F1 in db/db Mouse Model

The efficacy of fusion proteins in db/db mouse model (Institute of Model Animal Science, Nanjing University) was studied. A series of purified fusion proteins were diluted with 10 mM PBS during the experiment. The mice were randomly divided into four groups, including blank group (10 mM PBS), D3F1 group, D4F1 group and D5F1 group. Each db/db mouse was injected with 30 nM/kg fusion protein (Diluted with 10 mM PBS), the administration volume was 10 ml/kg, and 9 mice per fusion protein were injected once a week for two weeks. The results of sampling changes in blood glucose after administration are shown in FIG. 6. D3F1, D4F1 and D5F1 showed better hypoglycemic effects.

### Example 11: Study on the Lipid-Lowering Efficacy Effect of Dulaglutide, S1F1, D1F1 in db/db Mouse Model

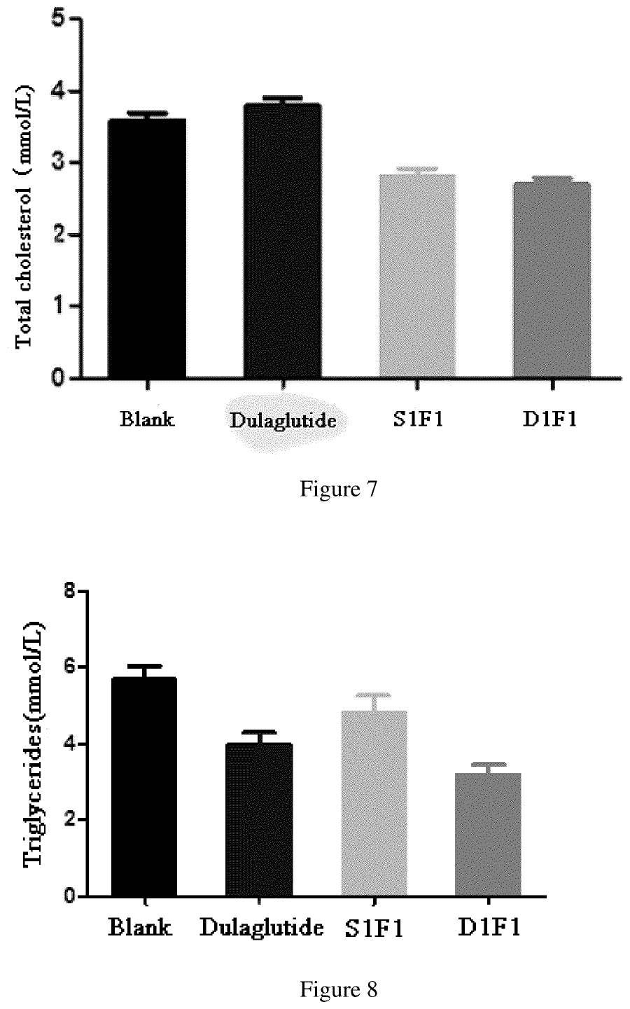

The db/db model mice (Institute of Model Animal Science, Nanjing University) were randomly divided into 4 groups, including blank group (10 mM PBS), dulaglutide group, S1F1 group and D1F1 group. Each db/db mouse was injected with 40 nM/kg fusion protein (Diluted with 10 mM PBS), the administration volume was 3 ml/kg, and 10 mice per fusion protein were injected once a week for two weeks. All mice were sacrificed 14 days after the initial administration, and their blood total cholesterol (TCHO) (see FIG. 7) and triglyceride (TG) content (see FIG. 8) were measured. The ability of D1F1 to lower total cholesterol and triglyceride content was higher than that of S1F1 group and dulaglutide group.

### Example 12: A Study of the Lipid-Lowering Efficacy of D1F2, D2F1 and D7F1 in db/db Mouse Model

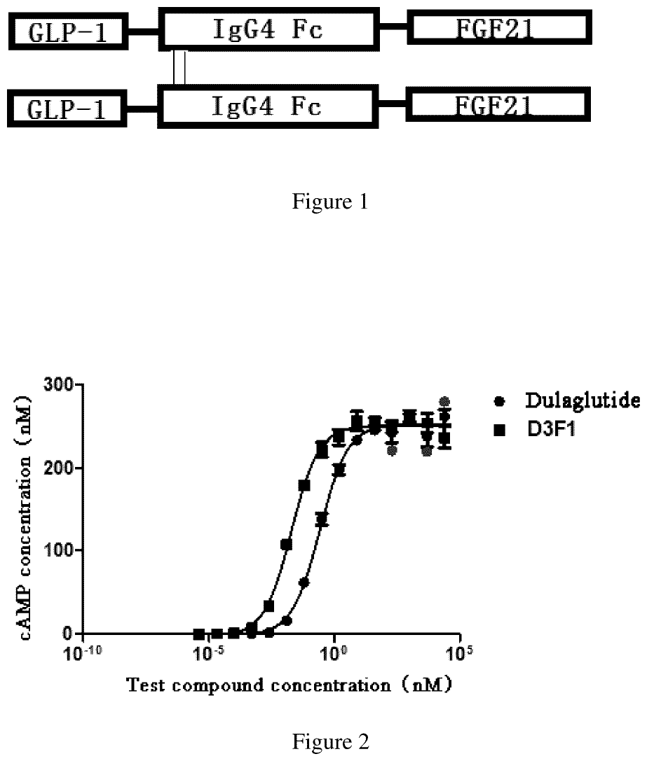

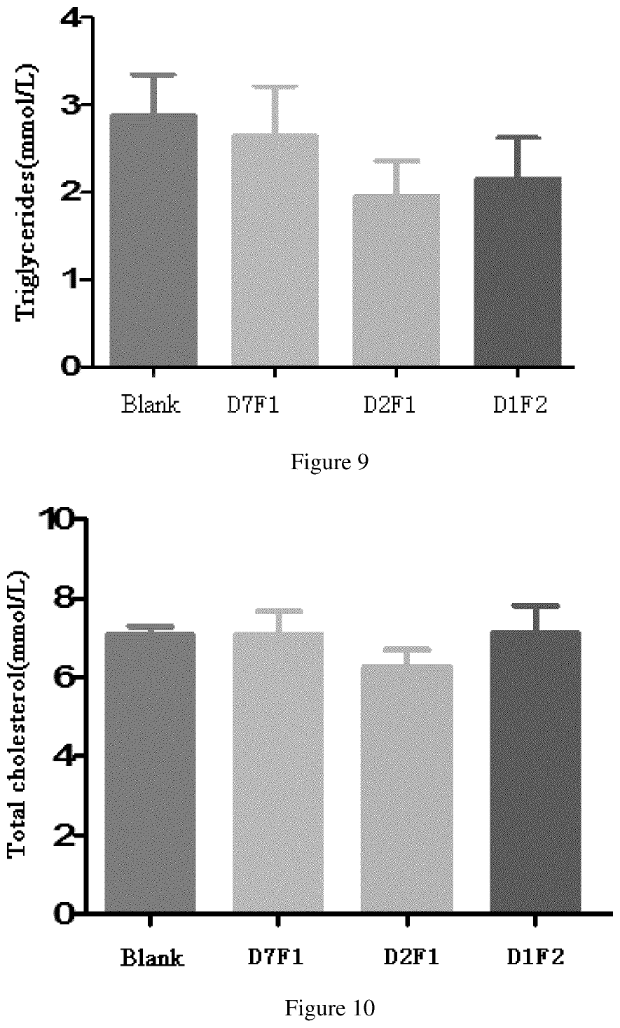

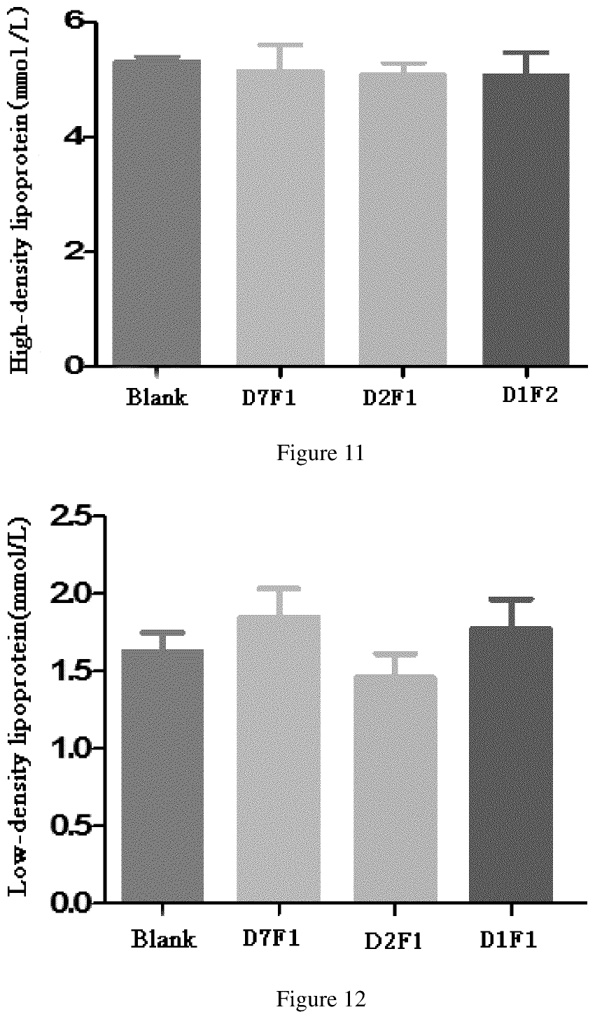

The db/db model mice (Institute of Model Animal Science, Nanjing University) were randomly divided into 4 groups, including blank group (10 mM PBS), D1F2 group, D2F1 group and D7F1 group. Each db/db mouse was injected with 24.5 nM/kg fusion protein (Diluted with 10 mM PBS), the administration volume was 10 ml/kg. Seven mice per fusion protein were injected once. The results of a serum triglyceride (TG) test after a single administration for 14 days are shown in FIG. 9; results of the total cholesterol (TC) content are shown in FIG. 1; results of high density lipoprotein (HDL-C) content are shown in FIG. 1, and results of the low density lipoprotein (LDL-C) content are shown in FIG. 12. The D1F2 and D2F1 groups showed a greater ability to lower triglycerides than D7F1 with a native FGF21 sequence. D2F1 showed stronger ability to lower triglyceride and total cholesterol than D1F2, which did not mutate at position 409 of IgG4 Fc.

### Example 13: A Study of Lipid-Lowering Efficacy of D3F1, D4F1, D5F1 in db/db Mouse Model

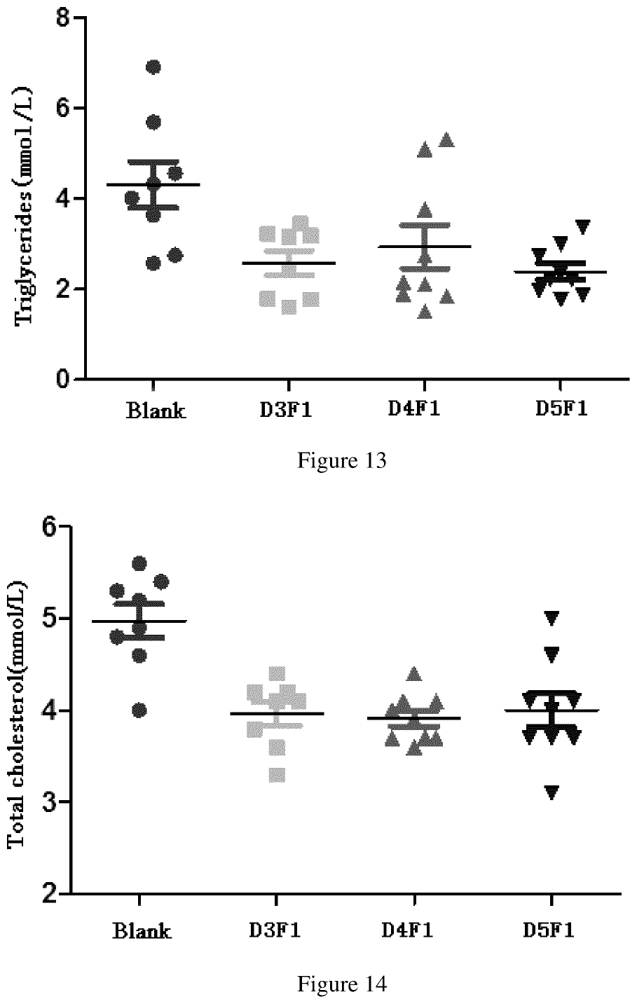

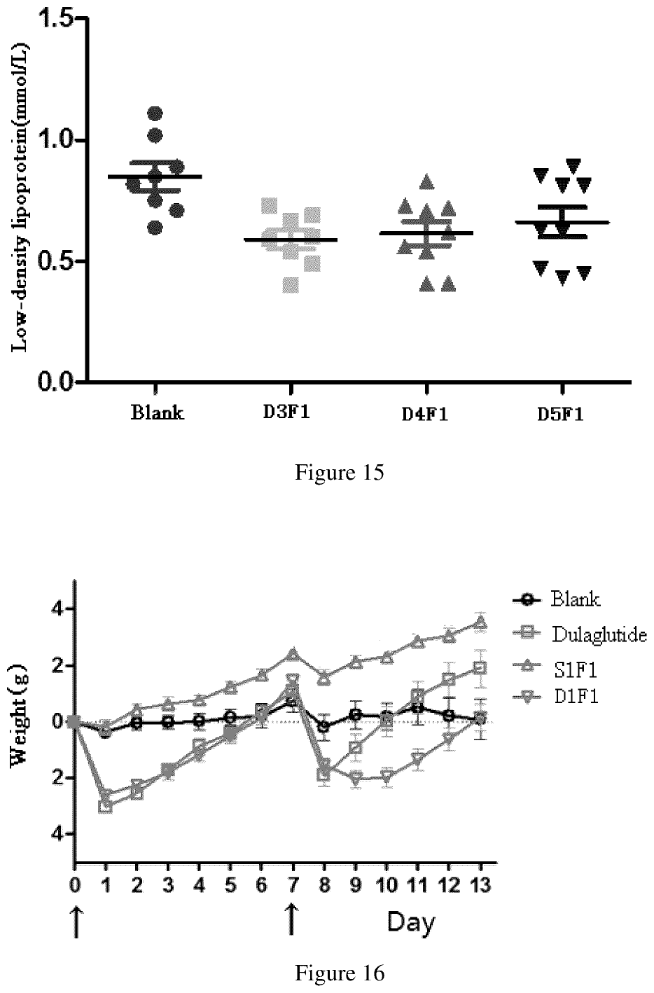

The db/db model mice (Institute of Model Animal Science, Nanjing University) were randomly divided into 4 groups, including blank group (10 mM PBS), D3F1 group, D4F1 group and D5F1 group. Each db/db mouse was injected with 30 nM/kg fusion protein (Diluted with 10 mM PBS), the administration volume was 3 ml/kg, and 9 mice per fusion protein were injected once a week for two weeks. Serum triglyceride (TG) results after 14 days of first administration are shown in FIG. 13. The total cholesterol (TCHO) results are shown in FIG. 14. Low-density lipoprotein (LDL-C) results are shown in FIG. 15.

### Example 14: A Study of the Weight-Loss Efficacy of Dulaglutide, S1F1, D1F1 in db/db Mouse Model

The db/db model mice (Institute of Model Animal Science, Nanjing University) were randomly divided into 4 groups, including blank group (10 mM PBS), dulaglutide group, S1F1 group and D1F1 group. Each db/db mouse was injected with 40 nM/kg fusion protein (Diluted with 10 mM PBS), the administration volume was 3 ml/kg, and 10 mice per fusion protein were injected once a week for two weeks. Body weight was recorded daily after administration, and results are shown in FIG. 16.

### Example 15: Study on the Pharmacokinetic of Dulaglutide, D1F1

Male ICR mice (Hunan Si Lake) were divided into 2 groups, each group with 3 mice, and each was subcutaneously injected with 10 nmol/kg dulaglutide or 10 nmol/kg D1F1 (Diluted with 10 mM PBS). Venous blood was collected through retro-orbital venous plexus at 1, 5, 7, 24, 48, 96, 144, 192, 240, 288 h before administration and after administration respectively. The anti-GLP-1 antibody (Bioporto, CAT.NO. ABS 033-10) was used as the capture antibody, the anti-human IgG Fc antibody (Southern Biotech, CAT.NO. 9200-05) was used as the detection antibody, the pharmacokinetic parameters were determined by the ELISA method. The results are shown in Table 5.

TABLE 5

| | Tmax | Cmax | AUClast | MRTlast |
|---|---|---|---|---|
| Test substance | (h) | (ng/ml) | (h*μg/ml) | (h) |
| D1F1 | 6.33 | 3.77 | 149.6 | 32.47 |
| Dulaglutide | 5.67 | 1.72 | 46.9 | 37.56 |

The average plasma exposure (AUCₗₐₛₜ) and Cmax of D1F1 fusion protein were significantly higher than those of dulaglutide, indicating that absorption of the fusion protein can be increased after fusion with FGF21. MRTlast indicates the average retention time of drug molecules in the body, which were equivalent for both D1F1 and dulaglutide in mice.

### Example 16: A Study of the Pharmacokinetic of Dulaglutide, D5F1 in ob/ob Mouse Model

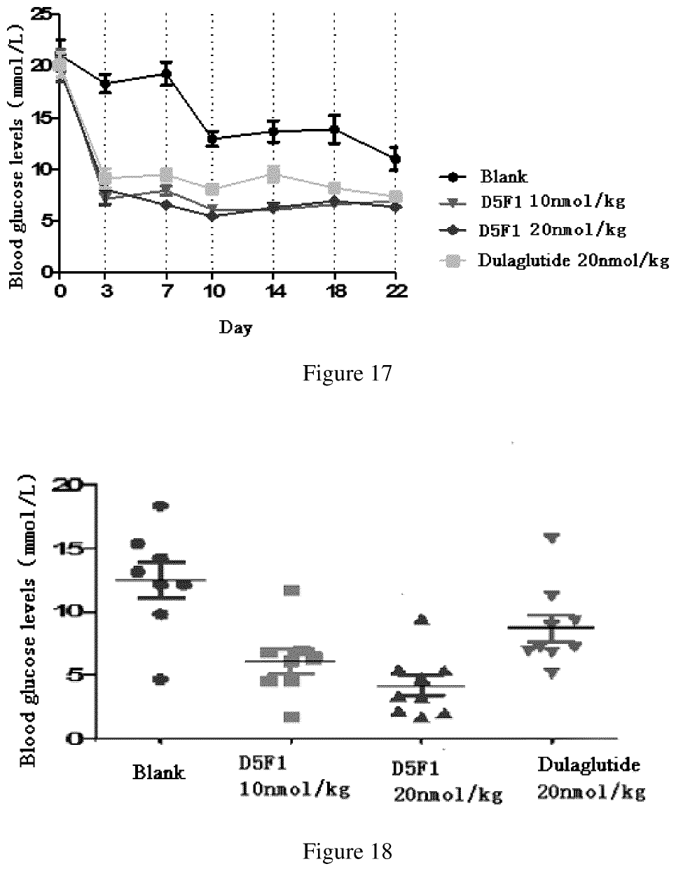

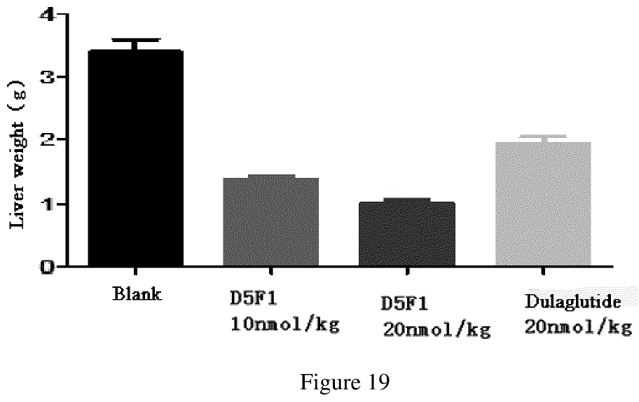

The ob/ob mice (Institute of Model Animal Science, Nanjing University) were randomly divided into 4 groups, including blank group (10 mM PBS), the D5F1 10 nmol/kg group, D5F1 20 nmol/kg group and dulaglutide 20 nmol/kg group (Diluted with 10 mM PBS). The administration volume was 3 ml/kg, and 9 mice per fusion protein were injected, twice a week for 3 consecutive weeks. The results of samples blood glucose testing before each administration are shown in FIG. 17. Compared with dulaglutide, D5F1 displayed a better hypoglycemic effect, and the hypoglycemic curve of the D5F1 group was more stable. Blood was taken from the orbit of the second day after the last administration for 16 hours, then serum was collected. Serum glucose was monitored (results shown in FIG. 18). After dissected, the liver was weighed (results shown FIG. 19). According to the results of liver weight, D5F1 group has better improvement on hepatic steatosis.

A copy of a sequence listing of SEQ ID Nos. 1-38 disclosed herein is submitted herewith as a separate part of the disclosure in XML format, which is incorporated herein by reference in its entirety. The XML file name is PP683US-CON_listing_v3. The XML file was created on Nov. 27, 2024 and its size is 79,491 bytes.

What is claimed is:

1. A fusion protein comprising or consisting of:
a) a first polypeptide, wherein the first polypeptide is glucagon-like peptide-1 (GLP-1) or an analogue thereof;
b) a second polypeptide, wherein the second polypeptide is an IgG4 Fc portion comprising the amino acid sequence of SEQ ID NO: 2, or a variant thereof; and
c) a third polypeptide, wherein the third polypeptide is fibroblast growth factor 21 (FGF21) comprises the amino acid sequence of SEQ ID NO: 6 or a variant thereof,
wherein the first polypeptide is linked to the second polypeptide via a first linker and the third polypeptide is linked to the second polypeptide via a second linker; and
wherein the variant of SEQ ID NO: 6 comprises one or more mutations at positions including 167, 175 and 179; wherein the amino acid mutation at position 167 is selected from the group consisting of S167H and S167C; wherein the amino acid mutation at position 175 is selected from the group consisting of R175L, R175H and R175P; and wherein the amino acid mutation at position 179 is selected from the group consisting of Y179S and Y179F.

2. The fusion protein of claim 1, wherein the GLP-1 analogue comprises or consists of the amino acid sequence of SEQ ID NO: 7
(HX1EGTFTSDVSSYLEX2QAAKEFIAWLX3X4GX5X6),
wherein X1 is selected from G or V;
X2 is selected from E or G;
X3 is selected from V or K;
X4 is selected from K or N;
X5 is selected from G or E; and
X6 is selected from G, P or absent.

3. The fusion protein of claim 1, wherein the GLP-1 analogue comprises or consists of the amino acid sequence of SEQ ID NO: 1.

4. The fusion protein of claim 1, wherein the FGF21 variant further comprises at least one amino acid mutation at position 170 or 172, wherein
the amino acid mutation at position 170 is selected from the group consisting of G170E, G170A, G170C, G170D, G170N, and G170S; and
the amino acid mutation at position 172 is selected from the group consisting of S172L, S1721, S172V, S172A, and S172M.

5. The fusion protein of claim 4, wherein the FGF21 variant comprises at least one amino acid mutation at position 170 or 172, wherein
the amino acid mutation at position 170 is selected from the group consisting of G170E, G170A, G170D, and G170S; and
the amino acid mutation at position 172 is selected from the group consisting of S172L, S172V, and S172M.

6. The fusion protein of claim 4, wherein the FGF21 variant comprises at least one amino acid mutation at position 170 or 172, wherein
the amino acid mutation at position 170 is selected from the group consisting of G170E, and G170A; and
the amino acid mutation at position 172 is S172L.

7. The fusion protein of claim 1, wherein the FGF21 variant further comprises one or more mutations at one or more sites selected from the group consisting of 45, 100, 150, 151, 152, and 180; wherein
the amino acid mutation at position 45 is selected from the group consisting of A45K, A45R, A45E, and A45Q;
the amino acid mutation at position 100 is LIO0K;
the amino acid mutation at position 150 is P150A;
the amino acid mutation at position 151 is G151A;
the amino acid mutation at position 152 is I152V; and
the amino acid mutation at position 180 is selected from the group consisting of A180S, A180E, and A180G.

8. The fusion protein of claim 1, wherein the FGF21 variant further comprises one or more mutations at one or more sites selected the group consisting of 45 and 180,
wherein the amino acid mutation at position 45 is selected from the group consisting of A45K, A45R, A45E, and A45Q; and the amino acid mutation at position 180 is selected from the group consisting of A180S, A180E, and A180G.

9. The fusion protein of claim 1, wherein the amino acid mutation at position 167 of the FGF21 variant is S167H; the amino acid mutation at position 175 of the FGF21 variant is R175L; and the amino acid mutation at position 179 of the FGF21 variant is Y179F.

10. The fusion protein of claim 1, wherein the first linker and the second linker each independently comprises a polypeptide of 5 to 50 amino acid residues, and the polypeptide comprises at least 50% G.

11. The fusion protein of claim 1, wherein the first linker and the second linker each independently comprises a G-rich polypeptide comprising at least 50% G.

12. The fusion protein of claim 1, wherein the first linker and the second linker each independently is selected from the amino acid sequence of SEQ ID NO: 32 (GGGGSGGGGS), or SEQ ID NO: 13 (GGGGSGGGGSGGGGS), or SEQ ID NO: 33 (GGGGSGGGGSGGGGSA).

13. The fusion protein of claim 1, wherein the fusion protein is a homodimeric fusion protein or a non-homodimeric fusion protein.

14. A pharmaceutical composition comprising the fusion protein of claim 1, and optionally one or more pharmaceutically acceptable adjuvants.

15. A method of treating a metabolic disease in a patient comprising administrating a therapeutically effective amount of the fusion protein of claim 1 to the patient, wherein the metabolic disease is diabetes or hepatic steatosis.

16. A fusion protein comprising or consisting of:
a) a first polypeptide, wherein the first polypeptide is glucagon-like peptide-1 (GLP-1) or an analogue thereof;
b) a second polypeptide, wherein the second polypeptide is an IgG4 Fe portion comprising the amino acid sequence of SEQ ID NO: 2 or a variant thereof; and
c) a third polypeptide, wherein the third polypeptide is fibroblast growth factor 21 (FGF21) comprising a variant of the amino acid sequence of SEQ ID NO: 15,
wherein the first polypeptide is linked to the second polypeptide via a first linker and the third polypeptide is linked to the second polypeptide via a second linker; and
wherein the variant of SEQ ID NO: 15 comprises one or more amino acid mutations at positions 98 and 171; wherein the amino acid mutation at position 98 is selected from the group consisting of L98D and L98R; and wherein the amino acid mutation at position 171 is selected from the group consisting of P171A and P171G; and
wherein the variant of SEQ ID NO: 15 further comprises one or more mutations at positions including 167, 175 and 179; wherein the amino acid mutation at position 167 is selected from the group consisting of S167H and S167C; wherein the amino acid mutation at position 175 is selected from the group consisting of R175L, R175H and R175P; and wherein the amino acid mutation at position 179 is selected from the group consisting of Y179S and Y179F.

17. A pharmaceutical composition comprising the fusion protein of claim 16, and optionally one or more pharmaceutically acceptable adjuvants.

18. A method of treating a metabolic disease in a patient comprising administrating a therapeutically effective amount of the fusion protein of claim 16 to the patient, wherein the metabolic disease is diabetes or hepatic steatosis.
